(19) United States
(12) Patent Application Publication
(10) Pub. No.: US 2009/0234225 A1
(43) Pub. Date: Sep. 17, 2009

(54) FLUORESCENCE DETECTION SYSTEM

(75) Inventors: Edward W. Martin, Delaware, OH (US); Ronald X. Xu, Columbus, OH (US); Duxin Sun, Ann Arbor, MI (US); Stephen P. Povoski, Dublin, OH (US); Joseph Pierre Heremans, Upper Arlington, OH (US); Robert Lee, Hilliard, OH (US); Vish V. Subramaniam, Westerville, OH (US); Claudia Turro, Powell, OH (US)

Correspondence Address: STANDLEY LAW GROUP LLP, 6300 Riverside Drive, Dublin, OH, 43017, US

(73) Assignee: The Ohio State University Research Foundation, Columbus, OH (US)

(21) Appl. No.: 12/352,408

(22) Filed: Jan. 12, 2009

Related U.S. Application Data
(60) Provisional application No. 61/020,345, filed on Jan. 10, 2008.

(51) Int. Cl. A61B 6/00; A61K 49/00

(52) U.S. Cl. 600/431; 424/9.6

(57) ABSTRACT

Exemplary embodiments include systems, methods, and compositions for the intra-operative detection of target tissue. At least one embodiment includes a fluorescence detection instrument that may be used for intra-operative detection of a fluorescent targeting agent, its binding site, and its interaction within cancer tissues. An exemplary embodiment is highly sensitive to the local deposition of fluorescence agents even at a low concentration. In at least one embodiment, the system includes a handheld navigation instrument that is usable to excite, detect, and report the fluorescent deposition of the targeting agent in real-time. In alternative embodiments, the system includes a wearable unit to excite, detect, and visually report the fluorescent deposition of the targeting agent to the user. The wearable unit includes eyewear that allow the user to perform image-guided surgery based on the near real-time fluorescence detection of the fluorescent targeting agent.

### CROSS-REFERENCE TO RELATED APPLICATIONS

This non-provisional patent application claims the benefit of priority to U.S. Provisional Patent Application No. 61/020,345 filed Jan. 10, 2008, which is hereby incorporated by reference in its entirety.

The present invention was made with United States Government support under grant no. R01 CA120023 by the National Institute of Health. The United States Government may have certain rights to this invention under 35 U.S.C. §200 et seq.

### TECHNICAL FIELD

The disclosed embodiments of the present invention are in the field of intra-operative systems for detecting cancer, particularly in vivo detection systems utilizing fluorescent molecular targeting agents.

### BACKGROUND OF THE ART

The accurate assessment of surgical resection margins and the recognition of occult disease within adjacent peritumoral tissues and within regional lymph node basins during cancer surgery are important oncologic principles that minimize recurrence rates and ultimately translate into improvement of long-term patient outcomes. Existing modalities of cancer detection that are exploited for patients undergoing planned cancer surgery with a curative intent have generally focused and relied upon the preoperative acquisition of images, such as with computed tomography (CT), magnetic resonance imaging (MRI), and positron emission tomography (PET). However, such a preoperative approach fails to provide the oncologic surgeon with real-time, dynamic intraoperative information that may significantly impact on critical decision-making within the operating room.

Intraoperative detection systems that specifically integrate the concept of handheld probes as the specific acquisition unit are known. Those systems provide the oncologic surgeon with real-time information to more precisely locate occult disease within adjacent peritumoral tissues and within regional lymph node basins, as well as improve our current ability to accurately assess surgical resection margins. Intraoperative detection systems have the potential for broad-based application for malignancies in many organ systems.

One such system is Radioimmunoguided surgery (RIGS). RIGS for the intraoperative identification and resection of tumor-bearing tissue in both colorectal and pancreatic cancer patients has demonstrated efficacy. The RIGS concept utilizes radioactive-labeled (I¹²⁵) monoclonal antibodies along with a hand-held gamma detector to provide the oncologic surgeon with critical, real-time information within the operating room. The successful development of this hand-held gamma instrument detector system allows for more accurate detection and subsequent complete resection of such occult tumor-bearing tissues and lymph nodes containing these radioactive-labeled monoclonal antibodies. That system has been previously shown to significantly improve disease-specific survival. Despite the success of RIGS in such clinical trials, the handling and disposal of the radioactive-labeled material, specifically I¹²⁵, has remained a major stumbling block to the widespread acceptance and implementation of RIGS technology.

In order to eliminate the concerns with the handling and disposing of radioactive material, there is an unmet need for an intraoperative detection system that retains the functional benefits of the RIGS technology without the handling and disposal issues associated with radioactive-labeled material.

### SUMMARY

This and other unmet needs of the prior art are met by the system and method as described in more detail below.

Exemplary embodiments of the current system include fluorescent targeting agent compositions, excitation and detection equipment, and methods necessary for the detection of neoplasms in many organ systems.

Exemplary embodiments comprise a system for detecting a target tissue in an animal, comprising: a molecular targeting agent operably linked to a fluorescent unit; and a fluorescence detection instrument comprising: a light source emitting an excitation radiation capable of exciting the fluorescent unit across intervening biological tissue; and a detection unit capable of detecting fluorescent emissions from the fluorescent unit across the intervening biological tissues.

An exemplary embodiment includes a system for real-time optical detection of cancer cells. This embodiment comprises a fluorescent targeting agent and a fluorescence excitation, detection, and navigation instrument. In operation, the system locates tumor cells using novel fluorescent molecular targeting agents. An exemplary embodiment will detect tumors of less than 1 mm with only several hundred cancer cells.

An exemplary embodiment provides novel fluorescent tumor targeting agents that comprise a molecular targeting unit coupled to a fluorescent unit. In particular, the embodiment provides targeting units comprising at least one motif that is capable of locating both tumor endothelium and tumor cell mass. Before detection, the targeting unit will be associated with at least one fluorescent species. The phrase “operably linked” is used to refer broadly to any useful coupling between a targeting unit and the fluorophore. Such fluorescent targeting units are therapeutically and diagnostically useful, especially in the treatment and diagnosis of cancer, including metastases. More specifically, the fluorescent targeting agents of an exemplary embodiment, when used in conjunction with the appropriate fluorescence detection equipment provide the user with real-time information to more precisely locate occult disease within adjacent peritumoral tissues and within regional lymph node basins, as well as improve the assessment of surgical resection margins.

In at least one embodiment, a novel hand-held instrument is disclosed for the intra-operative detection of occult tumor and for the accurate assessment of surgical margins. In conjunction with an exemplary fluorescent targeting agent disclosed herein, the instrument optically detects cancer cells.

In exemplary embodiments, the fluorophores (“fluors”) maybe chemically associated with the targeting agents. However, in certain embodiments, the association of the fluor and the targeting agent occurs only after the targeting agent becomes associated with the desired marker. A targeting agent is not strictly necessary, however, because in certain applications, the fluors themselves may independently associate with the tissues of interest by passive means (e.g., the injection of fluorescent dyes).

In order to detect fluorescence emissions from neoplasms located at interior regions of the tissue, the fluorophores will preferably be fine-tuned to a near-infrared wavelength where tissue transparency is optimal and tissue autofluorescence is minimal. In at least on embodiment, the targeting agent is conjugated to a fluorescent dye. In other embodiments, the targeting agent includes a fluorescent nanoparticle. In example embodiments, the agent has an emission wavelength between 550-1000 nm. More preferably, the agent has an emission wavelength between 650-900 nm. While fluorophores with excitation and emission wavelength in the NIR are preferred, acceptable results may be obtained from fluorophores that have excitation and emission spectra outside this window. For example, for specific applications fluorophores that have excitation bands in the UV and emission bands in the visible range or the IR may also provide useful information.

Fluorophores useful for the disclosed systems may encompass a wide range fluorescent species including fluorescent dyes, fluorescent nanoparticles, proteins genetically engineered to fluoresce, or any other fluorophore that may be detectable with the detection instruments disclosed herein. Specific embodiments may also include metabolites that fluoresce when activated by effector enzymes associated with the targeting agent.

Nearly all tumor specific antigens may be targeted by embodiments of the present system using antibodies or equivalents to those antigens. Furthermore, the targeting agent need not necessarily include an antibody. Equivalent targeting agents such as peptides, other monoclonal antibodies, lectins, or other truncated antibody variations would also make appropriate targeting agents. In an exemplary embodiment, humanized anti-TAG-72 monoclonal antibodies are utilized as the targeting agent.

To facilitate intraoperative detection, a hand-held optical instrument and a control unit for the detection of fluorescence from the above described novel fluorescent molecular targeting agent system are disclosed. Preferably, the optical detection instrument is equipped with a light source to provide the fluorescence excitation radiation that is capable of illuminating a relatively large area of tissue. The excitation light may also be provided by an external source. The handheld sensor head of an exemplary embodiment may be specifically designed for enhanced illumination and fluorescence collection efficiency.

In an exemplary embodiment a fluorescence detection instrument will be used for intra-operative detection of fluorescent tumor targeting agent, their binding sites, and interaction within cancer tissues. An exemplary embodiment is highly sensitive to the local deposition of fluorescence agents even at a low concentration. In at least one embodiment, the system includes a handheld navigation instrument that is usable to excite, detect, and report the fluorescent deposition of the targeting agent in real-time. In alternative embodiments, the system includes a wearable unit to excite, detect, and visually report the fluorescent deposition of the targeting agent to the user. The wearable unit includes eyewear that allow the user to perform image-guided surgery based on the near real-time fluorescence detection of the fluorescent targeting agent.

The system of at least one exemplary embodiment produces a detectable signal to allow the user to detect the fluorescent targeting agent. In some embodiments, the instrument transmits an audible alert when the detected fluorescence intensity is greater than the specified threshold. In an alternative embodiment, the instrument transmits a visual signal, such as a blinking indicator light, for the same. In yet another embodiment, the signal is tactile, such as a vibration. With regard to the alternative wearable detection unit, the user may actually view in vivo images of the detected fluorescence through provided eyewear at or near real-time. Of course, the signal may be conveyed to the user by any combination of the signal detection methods mentioned above or otherwise known in the art. Furthermore, the handheld unit and the wearable detection unit may be utilized individually or in conjunction.

The fluorescence detection apparatus of an exemplary embodiment has a large acceptance angle and high efficiency for fluorescence collection. In an exemplary embodiment, the apparatus is able to reject the ambient noise and the background excitation light. Optionally, the enclosure of the hand-held instrument may be made from biocompatible materials. Preferably, the instrument can be sterilized to prevent infections.

In operation, the targeting agents are administered to a patient in an effective amount. The agent specifically binds a marker produced by or associated with neoplastic tissue, e.g. cancer. Time is then permitted to elapse following the administration for the fluorophore-labeled targeting agent to preferentially concentrate the agent in any neoplastic tissue. Given sufficient time, unbound fluorophore-labeled targeting agent may be cleared so as to increase the ratio of photon emissions from neoplastic tissue to background photon emissions in the patient. After the time has elapsed, the patient is accessed with a fluorescence detection instrument for determining sites exhibiting accretion of the fluorophore locator by detecting with the instrument elevated levels of fluorescence emission at the neoplastic sites. Tissue exhibiting elevated levels of fluorescent emissions may be removed and subjected to gross visual analysis. Such sites may alternatively or additionally be subjected to histological analysis.

This system will provide the surgeon with intra-operative, real-time cancer-specific information to accurately assess surgical resection margins and precisely locate occult metastatic disease. Such a cancer-specific system offers the potential for use in the detection of malignancies in a wide range of organ systems.

Exemplary embodiments include a method for intra-operative detection of a neoplastic tissue in an animal, comprising: (a) administering to an animal an effective amount of a fluorescent targeting agent that specifically binds a marker produced by or associated with the neoplastic tissue; (b) pausing after the administering step (a) for a period sufficient to allow unbound agent to clear; (c) after the pausing step (b), locating the neoplastic tissue by optically accessing the animal with a fluorescence detection instrument; and (d) resecting the neoplastic tissue identified by the fluorescence detection instrument during the accessing step (c).

Another exemplary embodiment comprises a cancer surgical method for a human patient, comprising
identifying a tumor using preoperative imaging;
administering an effective amount of a microbubble or nanobubble encapsulated targeting agent through IV injection;
applying an ultrasound pulse on a local tissue to selectively release the targeting agent;
pausing after the applying step for a period sufficient to allow unbound agent to clear;
locating the neoplastic tissue by optically accessing the animal with a fluorescence detection instrument; and
resecting the neoplastic tissue identified by the fluorescence detection instrument during the locating step.

An exemplary embodiment provides therapeutic and diagnostic methods for the treatment and diagnosis of cancer, utilizing fluorescent targeting agents and fluorescent detection instruments according to the disclosed embodiments.

### BRIEF DESCRIPTION OF THE DRAWINGS

A better understanding of the exemplary embodiments of the invention will be had when reference is made to the accompanying drawings, wherein:

FIG. 1 is a schematic showing the preparation of an exemplary fluorescent targeting agent. In this case, the near-infrared fluorescent reagent Indocyanine green (ICG) may be readily conjugated with the desired targeting unit (e.g., the antibody HuCC49ΔC_{H}2).

FIG. 2 is a schematic showing the preparation of another exemplary fluorescent targeting agent. In this case, antibody is readily labeled with CyDye Monoreactive NHS esters by acetylating the primary amino group of the antibody.

FIG. 3 is a schematic showing the preparation of another exemplary fluorescent targeting agent. In this case, antibody is readily labeled with the fluorescent dye Cy7 by acetylating the primary amino group of the antibody

FIG. 4 shows the background fluorescence in untreated mice using four different fluorescence filters available for the IVIS imaging system.

FIG. 5A-5D shows the experimental results of murine in vivo fluorescent imaging study of colorectal cancer xenografts using the fluorescent targeting agent HuCC49deltaCH2-Cy7 conjugates. At each time point indicated, there were five experimental conditions examined using the ICG filter: (1) one blank untreated mouse; (2) one mouse treated with Cy7 alone (no antibody); (3) one mouse with A375 tumor xenograft and administered the fluorescent targeting agent HuCC49-Cy7; (4) one mouse preblocked with MuCC49 antibody but having the LS174T tumor xenograft and administered the fluorescent targeting agent HuCC49-Cy7; and (5) one mouse with LS174T tumor xenograft and administered the fluorescent targeting agent HuCC49-Cy7 (with no preblock antibody).

FIG. 6A is an exemplary method for one-step tumor targeting with direct conjugation of antibody and nanoparticle. FIG. 6A illustrates antibody conjugation to nanoparticle using mercapto acid.

FIG. 6B is another exemplary method for one-step tumor targeting with antibody conjugation to nanoparticle using amphiphilic triblock polymer.

FIG. 7 is a schematic of an alternative nanoparticle two step tumor targeting method utilizing two conjugates.

FIG. 8 is a diagram depicting a fluorescence excitation and detection system.

FIG. 9 shows a schematic of an alternative wireless handheld excitation and detection system.

FIG. 10 shows an alternative wearable fluorescence detection apparatus.

FIG. 11 is a schematic showing an exemplary process for targeted delivery of tumor-specific antibody-fluorophore conjugate for image-guided cancer surgery (e.g., breast cancer).

FIG. 12 is a schematic showing an exemplary surgical goggle for intra-operative fluorescence detection.

FIG. 13 is an image visualized using a fluorescence imaging system similar to the goggle system in FIG. 12, except that the image is projected to a computer monitor instead of a HMD.

### DETAILED DESCRIPTION

U.S. Pat. No. 4,782,840 (the disclosure of which is expressly incorporated herein by reference) discloses a much improved method for locating, differentiating, and removing neoplasms. Such technique utilizes a radiolabeled antibody and a portable radiation detection instrument which the surgeon can use intraoperatively in order to detect sites of increased radioactivity. Despite the success of RIGS® in such clinical trials, the handling and disposal of the radioactive-labeled material, specifically I¹²⁵, has remained a major stumbling block to the widespread acceptance and implementation of RIGS technology. Through the use of innovative fluorescence excitation and detection system, novel fluorescent targeting agents, and methods for utilizing the same, embodiments disclosed herein eliminate the major disadvantages of RIGS without compromising detection efficacy.

The use of near infrared (NIR) light, of wavelengths between 700 and 1000 nm, is by now quite established for biomedical imaging Frangioni, J. V., Curr Opin Chem. Biol. October; 7(5):626-34 (2003). It represents a compromise between light absorption, scattering, and also limits the autofluorescence of the tissues themselves, which decreases the resolution of a fluorescent tissue marker. Near infrared fluorescence spectroscopy is sensitive to the biochemical make-up of a given biological tissue. The biochemical sensitivity is achieved through both endogenous (intrinsic) and exogenous (extrinsic) agents. Endogenous agents include tissue chromophores (such as oxy and deoxy hemoglobin (Cope, The development of a near-infrared spectroscopy system and its application for non-invasive monitoring of cerebral blood and tissue oxygenation in the newborn infant. London: University College London, (1991)) and fluorophores (such as NADH and flavins). Exogenous agents include fluorophores with dye-antibody conjugates, dye-peptide conjugates, and molecular beacons. Achilefu et al., Technol Cancer Res Treat, 3: 393-409, 2004; Gurfinkel et al., Dis Markers, 19: 107-121 (2003).

Although endogenous fluorescence spectroscopy (also called “autofluorescence”) has been implemented clinically for the detection of skin cancer, cervical cancer, colon cancer, gastric cancer, and oral cancer, its clinical significance is limited by the overlap of the absorption spectrum with that of non-fluorescent chromophores and by the small penetration depth of the ultraviolet excitation.

In the near infrared region of 650 nm to 900 nm, light may penetrate several centimeters into biological tissue (A. Yodh and B. Chance, Physics Today, vol. 48, pp. 34-40, (1995)), enabling noninvasive detection of structural and functional abnormalities in tissue. Fluorescent markers with excitation and emission wavelength at near infrared region (NIR, 650-900 nm) usually have low background in biological system and may achieve deeper tissue penetration capability. While fluorophores with excitation and emission wavelength in the NIR are preferred, acceptable results may be obtained from fluorophore that have excitation and emission spectra outside this window. For example, for specific applications fluorophores that have excitation bands in the UV and emission bands in the visible range or the IR may also provide acceptable results.

Although expressly not limited by theory, photon migration in biological tissue may be modeled by the Radiative Transfer Equation below, with the assumption that wave phenomena and inelastic collisions are not considered (Welch et al., Optical-Thermal Response of Laser-Irradiated Tissue, New York: Plenum Press (1995)):

$$\frac{1}{c} \frac{\partial I ( \vec{r} , t , {\vec{e}}_{s} )}{\partial t} + {\vec{e}}_{s} \cdot ∇ I ( \vec{r} , t , {\vec{e}}_{s} ) + ( {μ}_{a} + {μ}_{s} ) I ( \vec{r} , t , {\vec{e}}_{s} ) = {μ}_{s} {\int }_{4 π} f ( {\vec{e}}_{s} , {\vec{e}}_{s}^{′} ) I ( \vec{r} , t , {\vec{e}}_{s} ) {d}^{2} {\vec{e}}_{s}^{′} + q ( \vec{r} , t , {\vec{e}}_{s} ) \qquad ( Eq . 1 )$$

Where c is the speed of light, t is time, \vec{e}ₛ is the direction vector, \vec{r} is the position vector, μₐ is the absorption coefficient, μₛ is the scattering coefficient, f (\vec{e}ₛ,\vec{e}ₛ′) is the scattering phase function, q(\vec{r},t,\vec{e}ₛ) is the radiation source, and I(\vec{r},t,\vec{e}ₛ) is the energy transfer per unit time per unit solid angle through a unit area at position \vec{r} and time t.
The first two terms on the left side of the equation represent the advective derivative of optical energy flow in the medium. The last term on the left side is associated with the attenuation of the energy due to scattering and absorption of the medium. The first term on the right side of the equation is the energy increase due to multiple scattering effects of the photons, and the low term on the right is the optical energy deposition by light sources. In turbid (highly scattered) media such as biological tissue, diffuse approximation may be used and the above equation can be simplified into:

$$\frac{\partial }{\partial t} {Φ}_{d} ( \vec{r} , t ) = D {∇}^{2} {Φ}_{d} ( \vec{r} , t ) - {μ}_{a} v {Φ}_{d} ( \vec{r} , t ) + v S ( \vec{r} , t )$$

To estimate the penetration depth of the excitation light and the yield of the fluorescence emission, modified Beer-Lambert law and 1D fluorescence analysis may be used by further simplifying the above diffuse equation into one dimensional expressions.

Embodiments disclosed exploit the ability of these longer wavelength fluorescent emissions to penetrate the peritumoral tissue surrounding neoplastic cells. In preferred embodiments, the targeting agents function as a vehicle for delivering or anchoring detectable fluorophores to the site of interest. A “targeting agent” includes substances which preferentially concentrate at the tumor sites by binding with a marker (the cancer cell or a product of the cancer cell, for example) produced by or associated with neoplastic tissue or neoplasms. Appropriate targeting agents include antibodies. The term “antibody” is used broadly herein to refer to both polyclonal and monoclonal antibodies, antibody fragments, chimeric versions of whole antibodies and antibody fragments, and humanized versions thereof. It will be appreciated, however, that substances which mimic the function of antibodies in selectively concentrating at the sites of neoplastic tissue are also within scope of this invention, though such substances may not be subsumed within the traditional definition of “antibody”.

In some embodiments, the targeting agent may be a genetically expressed protein, such as a fluorescent protein, that may be preferentially expressed in the tissue of interest. Alternatively the targeting agent may be a fluorescent metabolite or a fluorescent dye which may become concentrated in a particular tissue of interest (e.g., cancer tissue).

The identification of antigens expressed by tumor cells and the preparation of monoclonal antibodies which specifically bind such antigens is well known in the art. Anti-tumor monoclonal antibodies exhibit potential application as both therapeutic and diagnostic agents. Such monoclonal antibodies have potential application as diagnostic agents because they specifically bind tumor antigens and thereby can detect the presence of tumor cells or tumor antigens in an analyte. For example, use of monoclonal antibodies which bind tumor antigens for in vitro and in vivo imaging of tumor cells or tumors using a labeled form of such a monoclonal antibody is conventional in the art. Essentially, if the monoclonal antibody is attached to a fluorophore, the monoclonal antibody functions as a vehicle, i.e. it directs the fluorophore moiety to a neoplasm that expresses the target antigen bound by the monoclonal antibody.

One specific tumor antigen against which various monoclonal antibodies have been developed is tumor-associated glycoprotein TAG-72. TAG-72 is expressed on the surface of various human tumor cells, such as the LS174T tumor cell line (American Type Tissue Collection (ATCC) No. CL188, a variant of cell line LS180 (ATCC No. CL187)), a colon adenocarcinoma line. Various research groups have reported the production of monoclonal antibodies to TAG-72. Many human cancer cells, including human breast cancer tissues, colorectal cancer tissues, and cancer cell lines (e.g., breast cancer cells MCF-7 and colon cancer cells LS174T), express high levels of TAG-72. Murine and humanized anti-TAG-72 antibodies localize and target more than 80% of breast, colorectal, and pancreatic cancer in humans. In an exemplary embodiment, the molecular targeting agent comprises a purified antibody that binds specifically to TAG-72.

One example thereof, CC49, is a murine monoclonal antibody of the IgG.sub.1 isotype. This monoclonal antibody is a second generation monoclonal antibody prepared by immunizing mice with TAG-72 purified using the first generation antibody B72.3. Colcher et al., Proc. Natl. Acad. Sci. USA, 78:3199-3203 (1981). CC49 specifically binds TAG-72, and has a higher antigen-binding affinity than B72.3. Muraro et al., Cancer Res., 48:4588-4596 (1988). This monoclonal antibody has been reported to target human colon carcinoma xenografts efficiently, and to reduce the growth of such xenografts with good efficacy. Molinolo et al., Cancer Res., 50:1291-1298 (1996); Colcher et al., J. Natl. Cancer Inst., 82:1191-1197 (1990). Also, radiolabeled CC49 has been reported to exhibit excellent tumor localization in several ongoing clinical trials.

While murine antibodies have applicability as therapeutic agents in humans, they are disadvantageous in some respects. Specifically, murine antibodies, because of the fact that they are of foreign species origin, may be immunogenic in humans. This may result in a neutralizing antibody response (human anti-murine antibody (HAMA) response), which is particularly problematic if the antibodies are desired to be administered repeatedly, e.g., in treatment of a chronic or recurrent disease condition. Also, because they contain murine constant domains they may not exhibit human effector functions.

In an effort to eliminate or reduce such problems, chimeric antibodies have been disclosed (for example, U.S. Pat. No. 6,753,152, expressly incorporated by reference herein). Chimeric antibodies contain portions of two different antibodies, typically of two different species. Generally, such antibodies contain human constant regions and variable regions of another species, typically murine variable regions. For example, some mouse/human chimeric antibodies have been reported which exhibit binding characteristics of the parental mouse antibody, and effector functions associated with the human constant region. See, e.g.: U.S. Pat. No. 4,816,567 to Cabilly et al.; U.S. Pat. No. 4,978,745 to Shoemaker et al.; U.S. Pat. No. 4,975,369 to Beavers et al.; and U.S. Pat. No. 4,816,397 to Boss et al.

In an exemplary embodiment disclosed herein, the humanized anti-TAG-72 antibody, HuCC49ΔC_{H}2 is utilized as the targeting agent (see U.S. Pat. No. 6,818,749; U.S. Pat. No. 7,256,004, expressly incorporated herein by reference). The antibody HuCC49ΔC_{H}2 was generated and tested for tumor targeting and pharmacokinetics in a pilot clinical trial in 21 patients with recurrent or metastatic colorectal cancer in RIGS®. HuCC49ΔC_{H}2 generated no HAMA response (<20 ng/ml) in all 55 patients pre- and post-injection at 4-12 weeks. J. Xiao et al., Cancer Biother Radiopharm, vol. 20, pp. 16-26, 2005. Tumor targeting of HuCC49ΔC_{H}2 was evaluated in tumors at various metastatic sites (liver, abdominal wall, lymph node, pelvis, kidney, pancreas, stomach, small intestine, and colon). The HuCC49ΔC_{H}2 levels in blood were monitored from day 1 to day 21. The ratio of tumor to blood of HuCC49ΔC_{H}2 levels were 5 to 10-fold higher from day 7 to day 21 post-injection. Because of its extended biological half-life, specific embodiments of the present invention utilize HuCC49ΔC_{H}2 antibody. The HuCC49ΔC_{H}2 levels in tumors and normal tissues were measured at the day of surgery. The ratio of tumor to normal tissues increased 9 to 40-fold. The pharmacokinetics suggest that the a phase elimination half-life of HuCC49ΔC_{H}2 from blood was 2 days.

An exemplary embodiment of the present invention comprises administering to a patient an effective amount of a fluorescent targeting agent which specifically binds a marker produced by or associated with neoplastic tissue. The dosage of fluorescent targeting agent is such that the fluorescence detection instrument can be utilized for determining neoplastic sites exhibiting accretion of the fluorescent targeting agent. Targeting agent dosages may depend upon the specific type of fluorophore used, the intensity of the excitation light source, the type of molecular targeting agent, the sensitivity of the detection equipment, and other factors which may affect dosage requirements as those skilled in the art will appreciate.

The immediate accession of the patient with the fluorescence detection instrument is not advisable. Preferably, time is permitted to elapse following administration of the fluorescent targeting agent in order for unbound fluorescent targeting agent to be cleared from the tissue surrounding the lymph nodes to be surveyed. Suitable fluorescent detection equipment functions by determining a level of fluorescent emissions over and above that normal background emissions found at the location (e.g., operating room) where the patient is being surveyed as well as the blood pool background (fluorescent targeting agent circulating in the blood stream), and surrounding tissue which may contain circulating unbound fluorescent targeting agent. The time may be as short as a few minutes on up to several weeks, depending upon how fast the patient's body clears (often metabolizes) the fluorescent targeting agent. Of importance is the recognition that the fluorescent targeting agent will be bound to the tumor cell site with its fluorophore intact, albeit at reduced levels, after such time period has elapsed.

Once the suitable interval has elapsed, the patient is accessed with the fluorescence detection instrument. The relevant sites are surveyed with the instrument for determining accretion of fluorescent targeting agent by detecting with the instrument elevated levels of fluorescent emissions at the relevant sites.

Additional fluorescence detection devices may be used as is necessary, desirable, or convenient. Detection instruments may be utilized in conjunction with a laproscope, mediastinoscope, or like specific instrument which suitably can be outfitted with a miniaturized fluorescence detection device which can be placed in immediate adjacency with the lymph node in order to determine accretion of fluorescent targeting agent. Regardless of the instrument or technique employed, the exemplary embodiments encompass all such instruments and techniques.

The methods and detection systems disclosed may be used in conjunction with other existing modalities of cancer detection and imaging such as computed tomography, MRI, etc. For example, preoperative images of the surgical site acquired by other imaging modalities may be used by this disclosed system for fluorescence imaging reconstruction in order to enhance the accuracy and the depth resolution for intraoperative fluorescence tumor detection.

The fluorescence detection compositions according to the disclosed embodiments may be administered systemically, non-systemically, locally or topically, parenterally as well as non-parenterally, e.g. subcutaneously, intravenously, intramuscularly, perorally, intranasally, by pulmonary aerosol, by injection or infusion into a specific organ or region, buccally, intracranically or intraperitoneally.

Amounts and regimens for the administration and detection of the fluorescent targeting agents according to the disclosed embodiments can be determined readily by those with ordinary skill.

### EXAMPLES

The following examples are included to demonstrate exemplary embodiments of the invention. It should be appreciated by those skilled in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Targeting Agents Utilizing Fluorescent Dyes

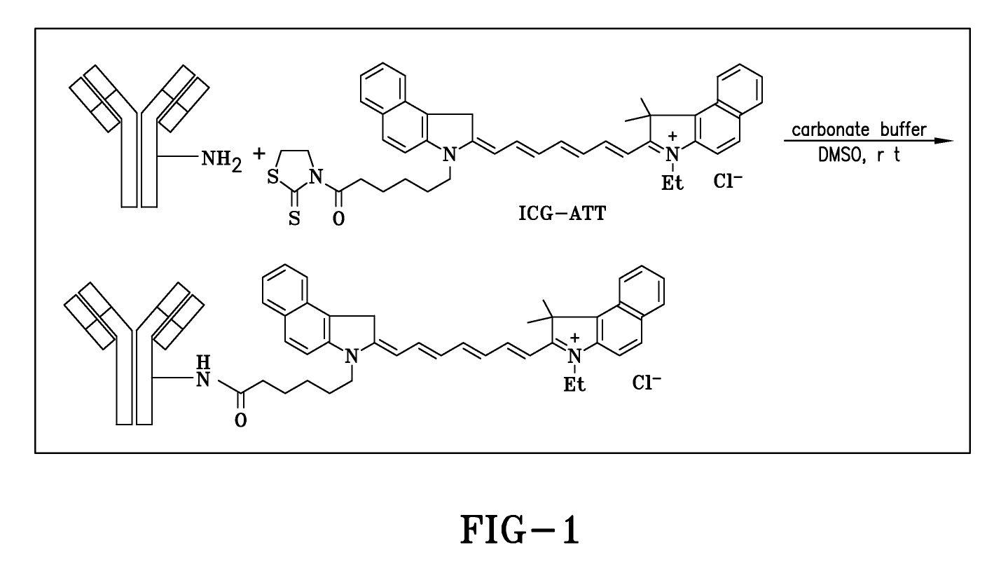

Referring to FIG. 1, an exemplary embodiment utilizing the fluorescent dye Indocyanine green (ICG) is shown. ICG is an FDA approved near-infrared fluorescent-labeling reagent in human. 3-indocyanine-green-acyl-1,3-thiazolidine-2-thione (ICG-ATT) readily reacts with the —NH₂ group on the antibody to form antibody-ICG conjugates. T. Hirata et al., Bioorg Med Chem, vol. 6, pp. 2179-84, 1998. Briefly, the selected antibody is treated with excess amount of ICG-ATT (the amount could be controlled base on the requirement of the conjugates) in the 0.1 M carbonate buffer (pH=9.5) and DMSO solution (4:1) for 30 min at room temperature. The unbounded dye was removed by gel filtration column chromatography (Sephadex LH-20). To remove the nonspecific and non-covalent binding of ICG to proteins, extraction method using ethyl acetate from the buffer solution will be performed. The antibody-ICG is soluble in the buffer solution while the ICG derivatives which non-covalently bind to proteins can be extracted to organic layer. After purification, the antibody-to-ICG ratio will be determined by comparing the absorbance of the conjugates with that of standard ICG DMSO solution using protein assay kit (Bio-Rad Inc., CA).

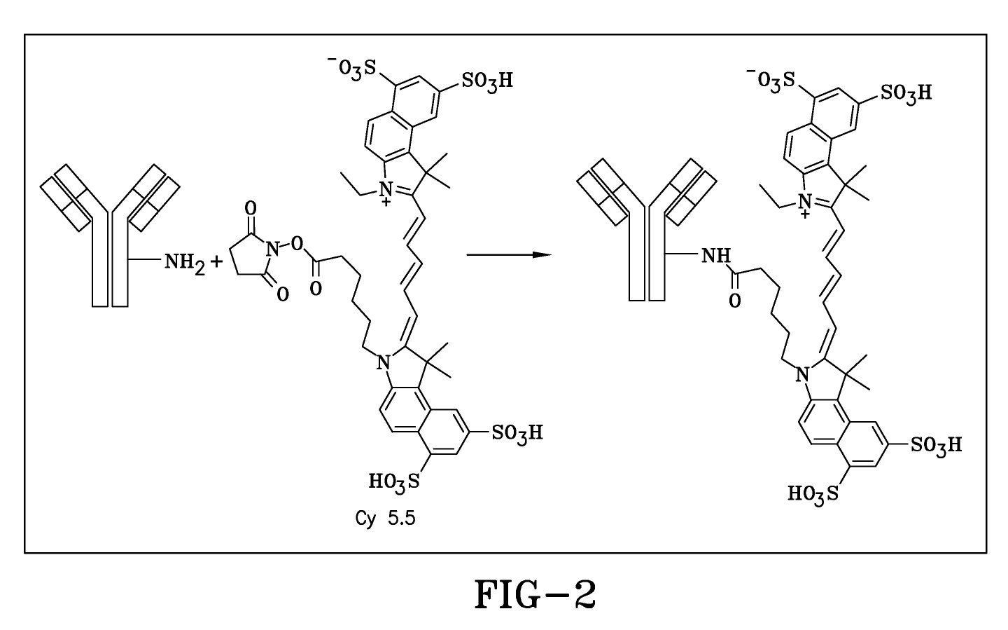

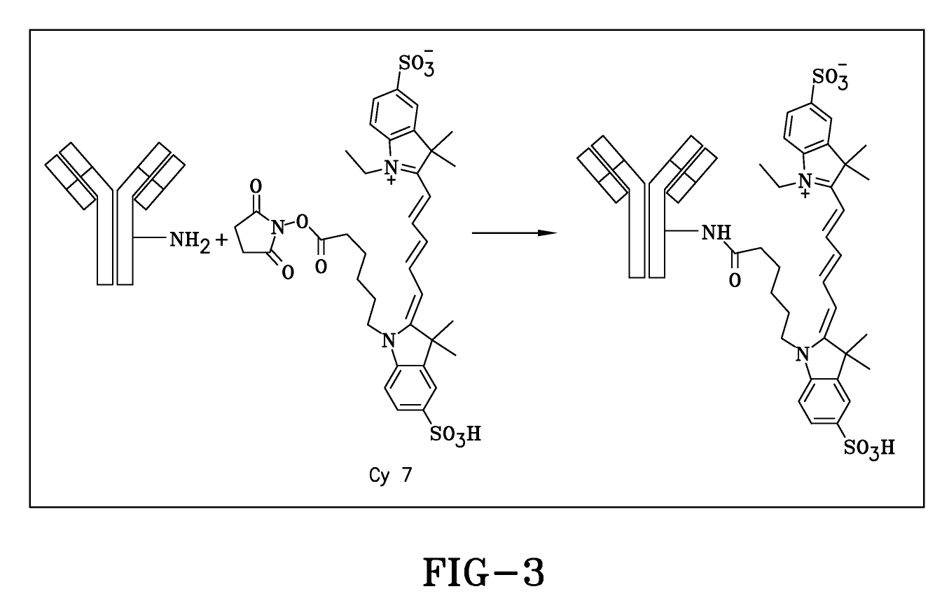

Other possible targeting agent-fluorophore conjugate is shown in FIGS. 2 and 3. As shown in FIGS. 2 and 3, the exemplary targeting antibody is readily labeled with CyDye Monoreactive NHS esters (such as Cy 5.5 and Cy7) by acylating the primary amino group of the antibody. The CyDyes are commercially available from GE Healthcare. When labeling HuCC49ΔCH2 antibody with NHS esters, the optimum conditions should be established by methods well known in the art. These include establishing the optimal ratio of CyDye NHS ester to protein and conjugation pH to give maximum fluorescence. R. B. Mujumdar et al., Bioconjug Chem, vol. 4, pp. 105-11 (1993). Preferably, the Cy5.5 NHS ester to antibody ratio will be approximately 20:1. The conjugation pH may be optimal at about 8.3.

HuCC49ΔC_{H}2 antibody (1 mg/ml) may be prepared in Na₂CO₃/NaHCO₃ buffer at pH 8.5. Cy5.5 mono NHS ester (0.5 mg) may be prepared in DMSO (200 μl). Cy Dye solution may be gradually added to the antibody solution with stirring. The solution may continue to be stirred for 60 minutes at room temperature in the dark. The CC49ΔCH2-Cy5.5 may then be purified by a Sephadex G-150 column. The column may be prewashed with 25 ml buffer pH 7.5, and then loaded with antibody-Cy 5.5 sample, and finally washed with the same buffer. The labeled CC49ΔCH2 may be eluted by water. The dye to antibody ratio can be calculated by the molar extinction coefficient at 280 nm (E²⁸⁰) of unlabeled HuCC49ΔC_{H}2 antibody and the molar extinction coefficient of Cy 5.5 (250000 M⁻¹cm⁻¹ at 675 nm).

### In-Vivo Fluorescent Imaging of Colorectal Cancer Xenografts with HuCC49deltaCH2-Cy7 Conjugates

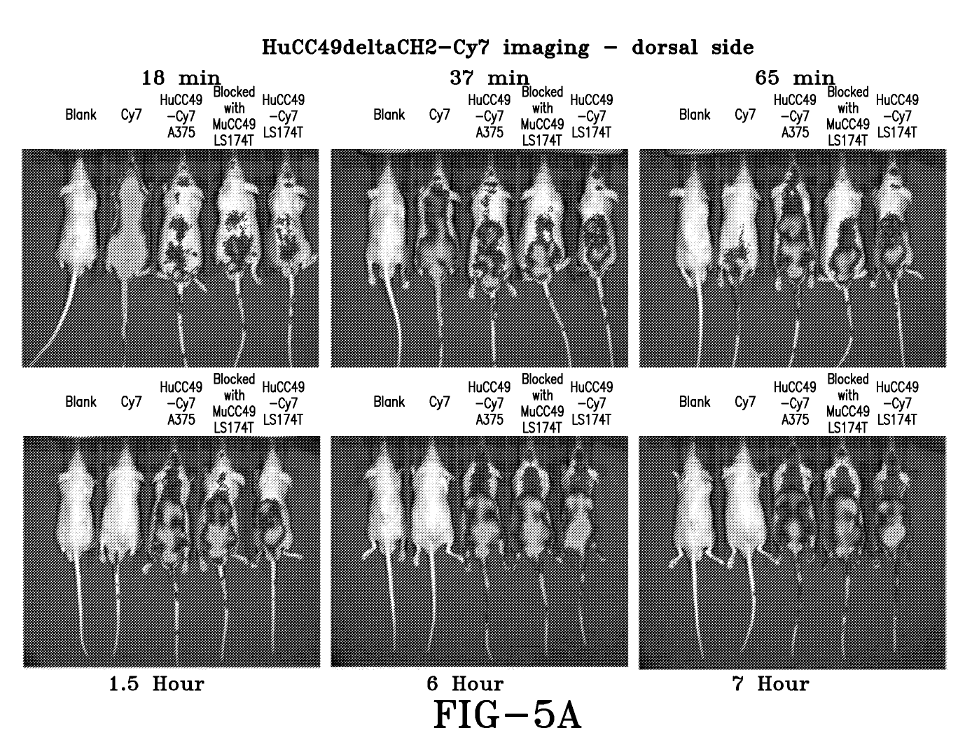

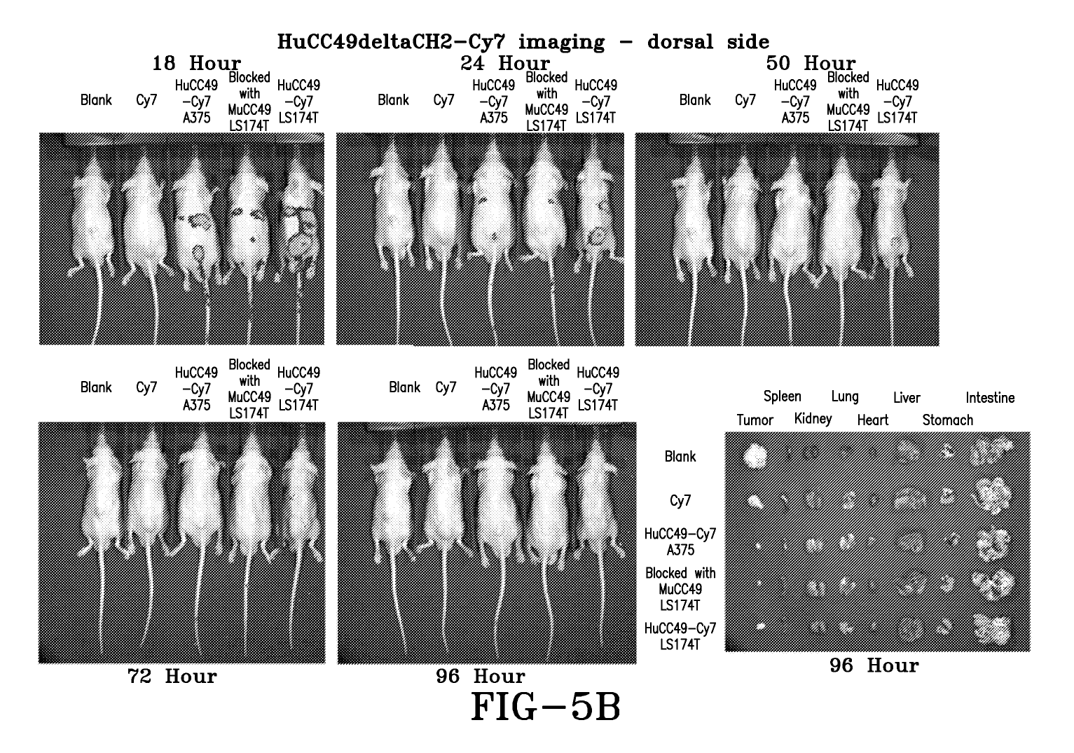

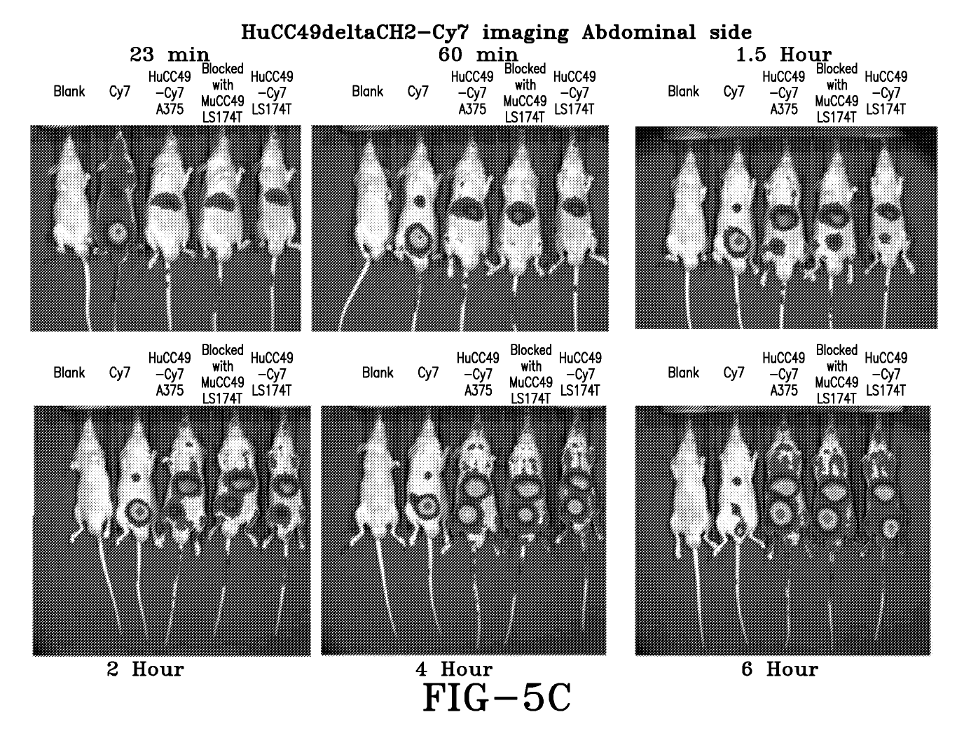

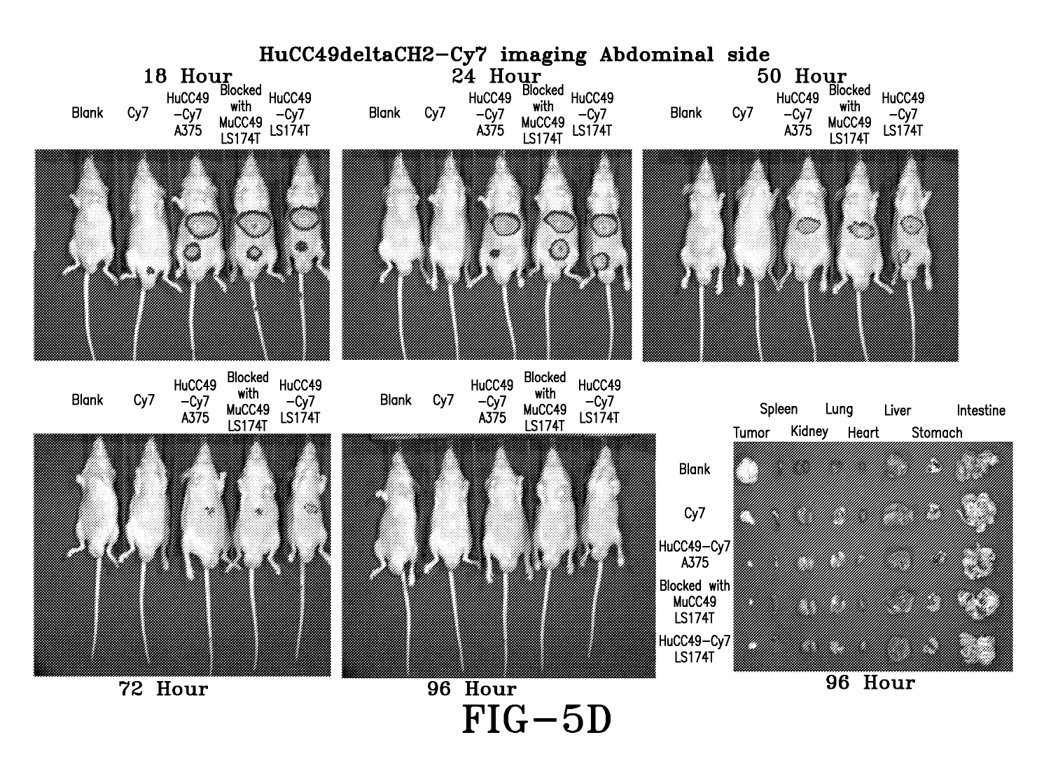

Referring to FIGS. 5A-5D, the following study was performed to demonstrate the safe and noninvasive use of fluorescent imaging agents to detect colorectal cancer xenografts in nude mice.

First, the targeting agent HuCC49deltaCH2-Cy7 conjugates were produced using the following protocol. A HuCC49ΔCH₂ antibody solution 2.8 ml (antibody 8.4 mg, 70 nmol) was prepared and adjusted to pH 8.9 by adding 150 μL Borate buffer (pH=10). Next, Cy 7 mono NHS esters (1 mg) were dissolved in DMSO (400 μl). Then, the volume needed to give the desired ratio of Cy Dye mono NHS ester to antibody was calculated (10:1). This Cy7 solution 230 μL (0.573 mg, 700 nmol) was gradually added to the antibody solution with stirring. The solution was stirred for an additional 2 hours at room temperature in the dark.

The labeled antibody was then purified by a PD-10 disposable column. The column was first pre-washed with 30 ml PBS. The sample was then loaded (3.2 ml) on the column and the eluent was discarded. The labeled antibody was then washed from the column with 3.5 mL PBS. The 3.5 mL eluent (green in color) was then collected in an Amicon Ultra-15 centrifugal filter (The centrifugal filter is pre-rinsed with 10 mL 0.1 N NaOH 5000 g×10 min and 10 mL PBS, 5000 g×10 min). The fraction (3.5 mL) was then concentrated by ultracentrifugation (Mᵣ cutoff 10 kD), 5000 g×±5 min. The concentrate was washed with PBS 10 mL×2 (each time 5000 g×20 min). To determine the purity of the labeled antibody, 5 μl of solution was injected onto a FPLC equipped with a Superdex 75 HR10/30 column. The elution profile was then taken with a 0.05 M phosphate buffer containing 0.15 M NaCl (pH 6) at a flow rate 1 ml/min. The UV-VIS detector wavelength was set at 280 nm and 747 nm.

To calculate D/P_{final} [Cy7]/[antibody]
(1) The molar extinction coefficient at 280 nm (E²⁸⁰) of unlabeled HuCC49ΔCH₂ antibody was determined (the antibody solution with known concentration was scanned against a blank at 280 nm using a UV-VIS spectrometer).
   The A²⁸⁰ of HuCC49ΔCH₂ (1.5 mg/mL) was measured as 2.28 (n=5). Hence of E²⁸⁰ HuCC49ΔCH₂ is determined as 182400 M⁻¹ cm⁻¹ (Assume MW of HuCC49ΔCH₂ as 120 kD).
(2) For Cy7, molar extinction coefficient of 200000 M⁻¹ cm⁻¹ at 747 nm is used. The calculation is corrected for the absorbance of the dye at 280 nm (approximately 11% of the absorbance at 747 nm).
   [Cy7 dye]=(A₇₄₇)/200000
   [antibody]=[A₂₈₀−(0.11*A₇₄₇)]/E²⁸⁰
   D/P_{final}=[dye]/[antibody]
   D/P_{final}=[E^{280 nm}*A₇₄₇]/{[A₂₈₀−(0.11*A₇₄₇)]*200000}
   A₇₄₇=3.28; A₂₈₀=2.56
   [Cy7 dye]=1.64×10⁻⁵ M
   [antibody]=(2.56-0.11*3.28)/182400=1.21×10⁻⁵ M=1.45 mg/mL
   D/P_{final}=1.36

Aliquots were stored at 4° C.

Tumor xenografts, LS174T cells were then implanted s.c. into the back of four 6-week-old male athymic nude mice (18-22 g, NCl) and A375 cells were implanted s.c. into the back of one 6-week-old female athymic nude mice (20 g, NCl). One mouse with LS174T tumor was randomly selected as a blank control without receiving any treatment. A volume of 180 μl HuCC49deltaCH2-Cy7 solution was transferred to an Eppendorf tube and diluted to 450 μl. One mouse with LS174T tumor was randomly selected and injected (tail vein) with 3 nmol (150 μl×2.46 mg/ml) MuCC49deltaCH₂ solution. Four hours later, the mouse was administrated 150 μl of diluted HuCC49deltaCH2-Cy7 solution. At the same time, another mouse with LS174T tumor and one mouse with A375 tumor were administered (tail vein) 150 μl of diluted HuCC49deltaCH2-Cy7 solution (1 nmol Cy7/mouse). A 10 nmol/ml Cy7 solution was prepared and 100 μl was injected into a mouse with LS174T tumor through tail vein.

Images were taken using ICG filter (exposure time 1s, high fluorescent level) at 0.3, 0.6, 0.8, 1, 1.33, 1.5, 1.75, 2, 2.5, 3, 4, 5, 6, 7, 18, 24, 50, 72 and 96 hours after injection. After 96 hours, the six mice were sacrificed. Additional fluorescent images of the dissected organs (tumor, spleen, kidney, lung, heart, liver, stomach and intestine) were obtained. The fresh organ tissues were stored in liquid nitrogen can.

The mice were fluorescently imaged using an IVIS imaging system. Four different fluorescent filters are available for this system; a GFP filter (ex 445-490 nm, em 515-575 nm); a DsRed filter (ex 500-550 nm, em 575-650 nm); a Cy5.5 filter (ex 615-665 nm, em 695-770 nm); and an ICG filter (ex 710-760 nm, em 810-875 nm).

The images of blank mice were obtained with the four filters.

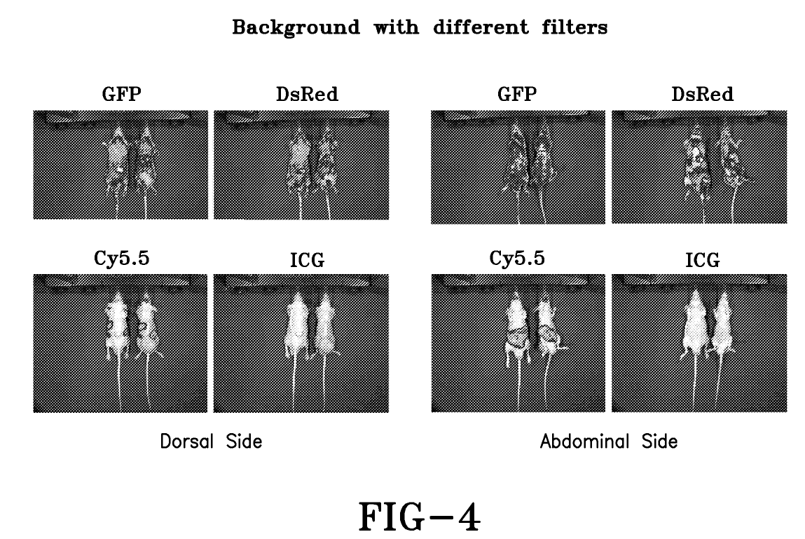

As demonstrated in FIG. 4, the background fluorescence was not detected under the ICG filter. Hence, the ICG filter was chosen for the imaging study. The fluor Cy7's excitation wavelength is at 730 nm and emission wavelength is at 770 nm, indicating that Cy7 can be detected with ICG filter.

FIGS. 5A-5D show the results from the imaging study using the IVIS system to detect in vivo fluorescence of the targeting agent. At each time point indicated, there were five experimental conditions imaged using the IVIS system equipped with the ICG filter: (1) one blank untreated mouse; (2) one mouse treated with Cy7 alone (no antibody); (3) one mouse with A375 tumor xenograft and administered the fluorescent targeting agent HuCC49-Cy7; (4) one mouse preblocked with MuCC49 antibody but having the LS174T tumor xenograft and administered the fluorescent targeting agent HuCC49-Cy7; and (5) one mouse with LS174T tumor xenograft and administered the fluorescent targeting agent HuCC49-Cy7 (with no preblock antibody).

As is apparent form FIGS. 5A-5D, the fluorescent targeting agent HuCC49deltaCH₂-Cy7 was successfully utilized to fluorescently detect colorectal cancer xenografts. The bottom right panel of FIG. 5B (dorsal view-tissues at 96 hours) and FIG. 5D (abdominal view-tissues at 96 hours) suggest that the targeting agent was mainly distributed in the in the liver and tumor, although fluorescence was also detected in kidney, spleen, stomach and bladder. The conjugates were finally eliminated from urine. Under the experimental conditions above, the maximum tumor/normal tissue ratio of HuCC49deltaCH2 was obtained between 18-24 hours after conjugate injection.

### Fluorescent Nanoparticles Targeting Agents

Organic NIR fluorescent markers may have limitations in their lifetimes (photobleaching), and it may be more problematic to control their absorption and emission wavelengths, which are strongly dependent on their chemical structure(52). As a result, inorganic nanoparticle embodiments may be appropriate as the fluorophore for the targeting agent for certain applications.

Commercial nanoparticle/antibody or nanoparticle/protein systems do exist (see, for example, Evident Technologies Troy, New York “EviFluors”, http://www.evidenttech.com). The existing nanoparticles are “core-shell” particles, consisting of two materials, one in the core of the particle, and the second one coating its surface. Typical particles have CdSe or InGaP cores, and a ZnS shell. The particles are typically 25 nm in diameter. They fluoresce at 520-620 nm wavelengths (CdSe/ZnS) and up to 680 nm (InGaP/ZnS). The wavelength of the photoluminescence of “type II” quantum dots can be pushed out to 850 nm (Kim et al., Nat Biotechnol, 22: 93-97, (2004)) when the core/shell structure is CdTe/CdSe, because of the particular way the conduction and valence bands align in that heterojunction system.

Disclosed here is a new and much more flexible nanoparticle system based on true infrared materials: the lead chalcogenides, typically lead sulfide (PbS), because their wavelengths can be extended from the visible wavelength range and far into the infrared range. PbS nanoparticles can be made to sizes down to 3.2 nm, where they absorb light at λ_{Ex}=800 nm and fluoresce at λ_{Em}=910 nm, optimal values for penetration depth, with suitably narrow absorption and emission peaks and a good separation between λ_{Ex} and λ_{Em}. Peterson and Krauss, Phys. Chem. Chem. Phys, 8:3851-3856 (2006). Furthermore, λ_{Ex} and λ_{Em} can be extended far into the infrared by increasing the size of the particles. This flexibility is the main reason why the initial focus of this proposal is on PbS nanoparticles.

Many synthesis techniques exist for PbS nanoparticles (see, for example, Peterson et al., Phys. Chem. Chem. Phys, 8: 3851-3856 (2006); Hines et al., Adv. Mater., 15: 1844-1849 (2003); Zhang et al Nanotechnology, 14: 443-446 (2003); Cademartiri et al., J Phys Chem B Condens Matter Mater Surf Interfaces Biophys, 110: 671-673 (2006)). Besides its advantages in optical penetration depth, PbS has four advantages over CdSe or CdTe from the point of view of quantum physics. Firstly, the intrinsic band gap between conduction and valence bands is smaller (Landolt-Bornstein Vol 17,Subvolume f: Springer Verlag, Berlin (1983)), so that optical activity can be extended farther into the infrared. Secondly, the band structure is such that electrons and holes are located in symmetrical conduction and valence bands and have nearly identical effective masses(Landolt-Bornstein Vol 17,Subvolume f: Springer Verlag, Berlin (1983)); in the Cd-chalcogenides, the holes have much heavier masses than the electrons. This implies that in PbS both electrons and holes undergo the effects of size-quantization in the nanoparticles; in the Cd-chalcogenides, the holes undergo very little size-quantization, only the electrons do. Therefore, the difference between the electron and hole sublevel energies, which dictate the optical transitions, vary much faster with the decrease in the nanoparticle sizes in PbS, resulting in a much larger range of tunability of λ_{Ex} and λ_{Em}. Thirdly, the effective masses of electrons and holes are smaller (82), which amplifies the abovementioned effect. In PbS, the energy levels in size-quantized nanoparticles have been shown experimentally to vary from 0.4 eV to 3.5 eV (83) (3000 to 354 nm), and, in another work, the absorption peak has been shown experimentally to be tunable from 1750 to 800 nm, with the fluorescence emission peak tunable from 1450 to 910 nm. Peterson et al., Phys. Chem. Chem. Phys, 8: 3851-3856, 2006. Finally, quantum-confinement arguments do not require a core/shell technology in PbS.

A second semiconductor alloy that may be prepared as nanoparticles are the lead-europium-telluride (Pb₁₋ₓEuₓTe) alloys first developed (United States Pat. No. 4,747,108) by the PI for use in quantum-well lead salts diode lasers. In this material system, the energy gap E_{g}, and consequently λ_{Ex} and λ_{Em} are a very strong function of the concentration x of Eu in the alloys. The tunability range is large, from E_{g}=0.26 eV (λ=4800 nm) in PbTe (Landolt-Bornstein Vol 17, Subvolume f: Springer Verlag, Berlin (1983)) to E_{g}=2.0 eV (λ=620 nm) in EuTe. Gosh et al., Phys. Rev., B 70: 115-211, (2004). A second advantage of this system is that wavelength tuning can be done by varying only x. Therefore, the particle size and the narrowness of the size distribution range become much less important.

Another embodiment includes oxides and sulfides doped or compounded with rare-earth elements. EuS has an energy gap of E_{g}=1.65 eV (Gosh et al., Phys. Rev., B 70: 115-211, (2004)) (A=750 nm), close to the optimal wavelength. EuO_{y}S_{1-y} alloys can be tuned between λ=750 and 1100 nm (Gosh et al., Phys. Rev., B 70: 115-211, (2004)), exactly the desirable A range. Additionally, yttrium-aluminum garnets (YAGs) are dopable with rare-earth elements (Nd, for instance), and the 4f level of the rare-earth atoms is at the basis of the optical properties. Suitable wavelengths can be found by changing the nature of the rare-earth dopant. YAGs are non-toxic.

### One Step Tumor Targeting by Direct Conjugation of Antibody and Nanoparticle

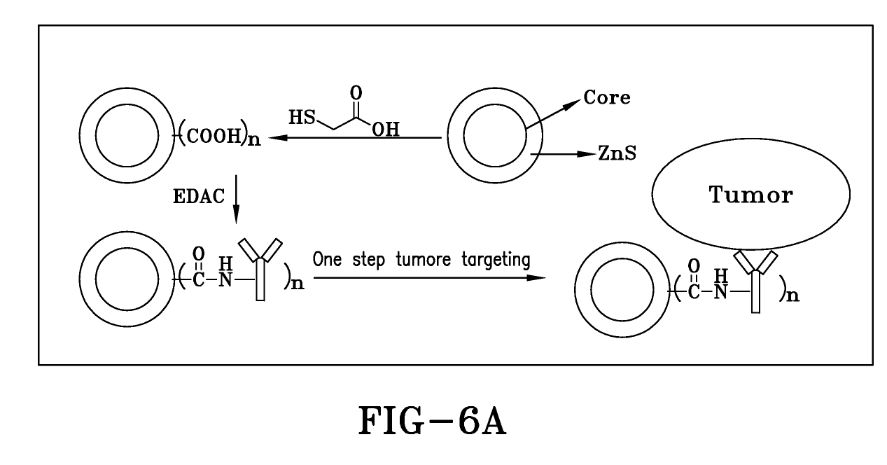

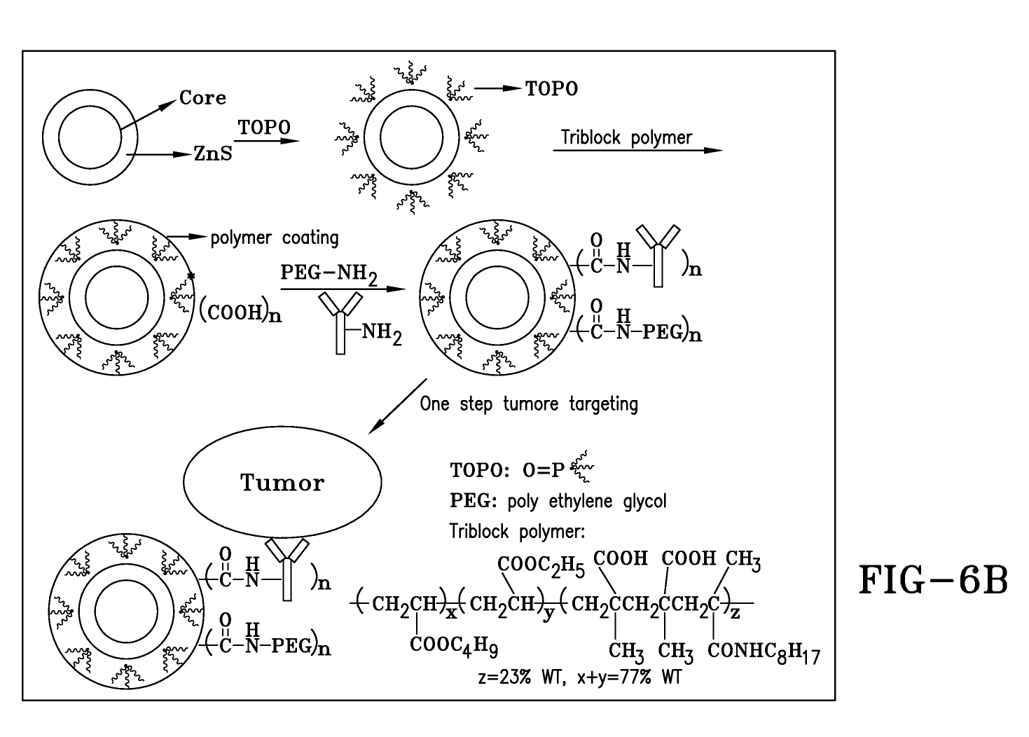

Referring to FIG. 6, in order to prepare the nonoparticles, the PbS or PbS/ZnS core-shell nanocrystals may be capped by trioctyl phosphine (TOP). They may then be mixed with modified poly(acrylic acid), which is synthesized by coupling a fraction (40%) of the carboxylic acid groups of a 1800 MW poly(acrylic acid) with octylamine using 1-ethyl-3-(3-dimethylaminopropyl)carbodiimide (EDC) in N,N-dimethylformamide. The surface coating will be cross-linked further by EDC (1-ethyl-3-(3-dimethylamino propyl)carbodiimide)-mediated coupling to lysine (or polyethylene glycol-lysine), and these materials will then be coupled to streptavidin or antibodies by an EDC-mediated coupling reaction. Wu et al., Nat Biotechnol, 21: 41-46 (2003).

In FIG. 6A, an exemplary method for conjugating the antibody is shown. In this example the PbS (or coated with ZnS) is modified with mercapto acetic acid in chloroform. The mercapto group should bind to a metal atom on the surface of the nanoparticle and leave carboxylic group free. The free carboxylic group provides not only higher water solubility but also availability for conjugating with bimolecular by forming covalent amide bond. Chan et al., Science, 281: 2016-2018 (1998). The conjugation may be done in the presence of 1-ethyl-3-(3-dimethylaminopropyl)carbodiimide (EDAC) and excessive of antibody (e.g., HuCC49ΔCH2). Using this method, the high and specific binding affinity of the antibody can be preserved. Furthermore, the mercaptoacetic acid layer can significantly reduce passive protein adsorption on nanoparticles and keep the nanoparticle bioconjugates as isolated entities.

In FIG. 6B an alternative amphiphilic triblock polymer method for conjugating antibody with nanoparticles is shown. In this method TOPO-capped nanoparticles are treated with an amphiphilic triblock polymer (Sigma) in chloroform/ethanol and followed by the activation using EDAC. Reaction of the activated nanoparticle with amino-PEG and then with the antibody in pH=8 buffer solution results in the formation of antibody-nanoparticle conjugates. The amphiphilic copolymer isolates the nanoparticles in vivo, and may reduce eventual nanoparticle toxicity. PEG improves biocompatibility and circulation. Gao et al., Nat Biotechnol, 22: 969-976, 2004.

### Two Step Tumor Targeting with Two Conjugates

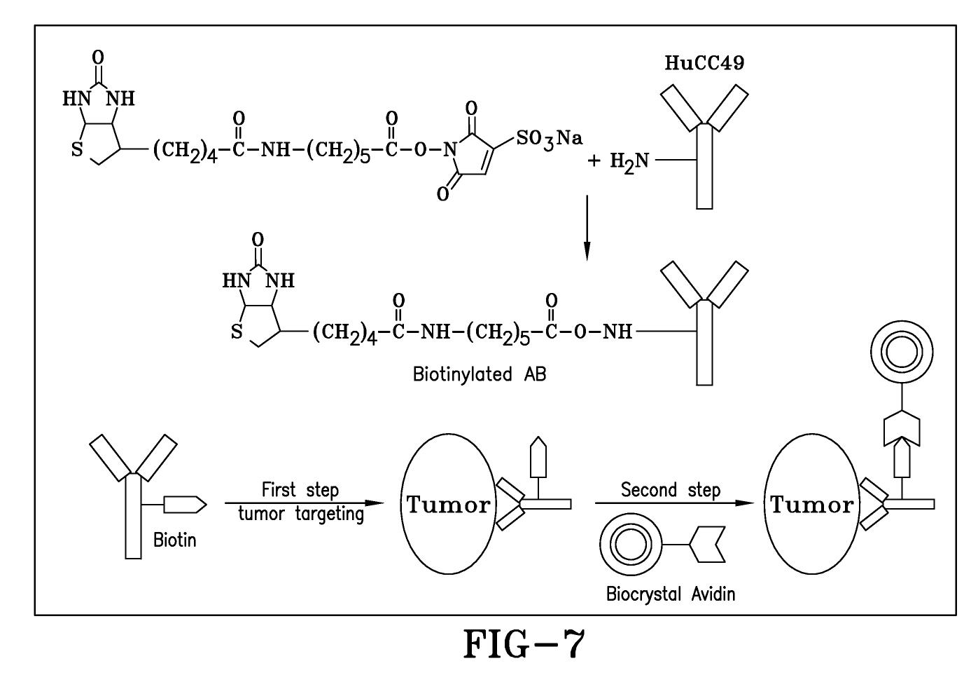

In the above one step targeting, the whole antibody-nanoparticle conjugate will be injected into animals. This procedure is certainly advantageous for direct tumor detection. But it has disadvantages such as its large molecular weight, and the necessity of re-evaluating the conjugates in addition to the antibody alone. To provide an alternative method, a two step targeting strategy to minimize the alteration of the antibody or peptide is demonstrated schematically in FIG. 7. In this method, the antibody will only be biotinylated. This modification should have minimal effects on the targeting ability of the given antibody.

In this method, Sulfo-NHS-biotin (PIERCE) may be used to biotinylate antibody (HuCC49ΔCH2) as shown in FIG. 7. The nanoparticle will be conjugated with avidin using the method described above. The tumor targeting will be achieved first with biotinylated antibody, then avidin conjugated nanoparticles will be applied. The biotin will bind to avidin for the localization of nanoparticle for tumor detection.

### Handheld Fluorescence Detection and Navigation Instruments

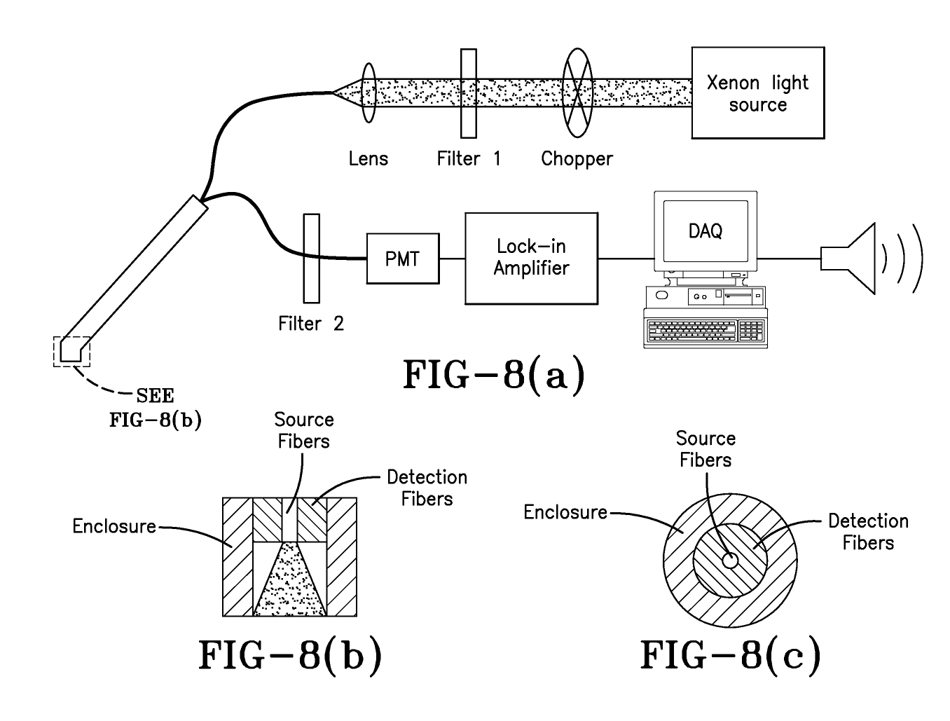

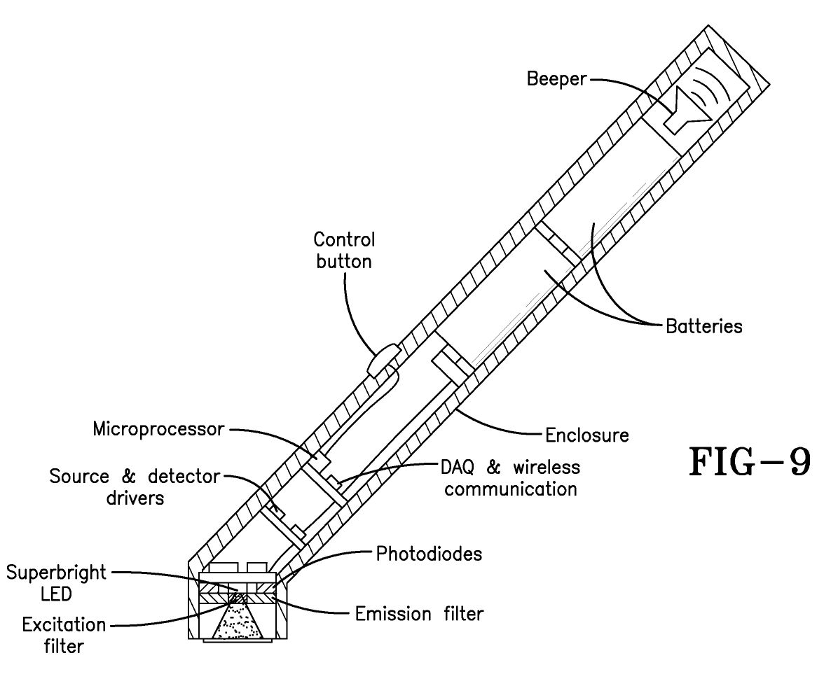

In order the detect the fluorescent targeting agents disclosed, exemplary embodiments of the handheld instrument for intraoperative detection of fluorescent tumor targeting molecules, their binding sites, and interaction within cancer tissues are shown in FIGS. 8 and 9.

Several instrument design criteria are apparent in the disclosed embodiments. First and most important is that the detection instrument should detect very small tumors. The second criterion is that the operation of the instrument must be as fast as possible since time in the operating room is of critical importance. The third criterion is simplicity of use. With these criteria, we disclose herein exemplary embodiments of fluorescence detection and navigation instruments useful for excitation, detection of the fluorescent targeting agents. An exemplary embodiment meets or exceeds all of the objectives above.

In at least one embodiment, the detection system comprises a hand held probe and a control unit. The control unit may be internal or external to the probe. The control unit includes a microcomputer that initializes the electronics, processes the signals, provides for diagnostics, and presents the output information to the user. It is important that the surgeon not be distracted from the surgical field to read meters or displays. For this reason, the preferred output is a sound that provides essentially a “yes” or “no” answer to the question of whether the probe is over tissue containing a statistically significant amount of fluorescent signal (i.e. cancer). A microprocessor maintains a running average of the count rate and makes a synthetic sound when the count rare exceeds the background count by a statistically significant amount, a user-set process called “squelching.” Preferably, the instrument makes a sound when the count rate is computed to be more than approximately 3 standard deviations (SD), or 3 sigma, above the 2-sec count rate for the normal tissue.

In the exemplary embodiment shown in FIG. 8, broadband light from a Xenon light source may be chopped, filtered and guided into source fibers. As shown, reflectance and fluorescence from biological tissue may be collected by detector fibers (FIGS. 8(b) and 8(c)). The fluorescence component may be picked up by a bandpass filter (see Filter 2, FIG. 8(a)), collected by a photomultiplier tube (PMT), and further amplified by a lock-in amplifier. The signal may then digitalized by a data acquisition card installed in a computer. The optical chopper and the lock-in amplifier effectively eliminate the ambient noise. The single photon counting capability offered by PMT may significantly enhance the detection sensitivity. Source fibers and detection fibers may be bundled together and placed in a stainless steel tubing (e.g., type 316) enclosure for biocompatibility and for ease of sterilization.

As shown in FIG. 8(b), the handheld sensor head is specifically designed for enhanced illumination and fluorescence collection efficiency and for reduced radiation hazard. In this embodiment, the source and the detection fibers may be recessed from the top of the sensor head so that the excitation light can illuminate a relatively large area of tissue. By using a large illumination area and by modulating the excitation light, the effective tissue radiation exposure is significantly reduced. The recessed detector fibers also enhance the fluorescence collection efficiency. Although not shown in FIG. 8 or 9, further detection efficiency enhancements may be achieved by beveling the fiber optical surfaces for fluorescence emission collection. Utzinger et al., J Biomed Opt, vol. 8, pp. 121-47 (2003).

An alternative wireless handheld embodiment is shown in FIG. 9. In this design, all the opto-electronic components may be hermetically sealed inside a handheld biocompatible tubing enclosure. Light, that may be provided by a super bright LED for example, is expanded to illuminate the area of detection. Fluorescence emission from the cancer tissue is filtered by an emission filter and collected by photodiodes. The fluorescence emission is digitalized and further processed by an embedded microprocessor. Upon detection of high deposition of fluorescence agent, the detector may trigger a beeper or another detection signal. Collected data can be sent real time to a host computer through a wireless communication port. The handheld instrument embodiment shown in FIG. 9 is portable, it may be manufactured at low cost, and it may be made disposable as an intraoperative cancer detection tool for surgical oncologists.

### Image-Guided Surgery using Wearable Fluorescence Detection and Navigation Instruments

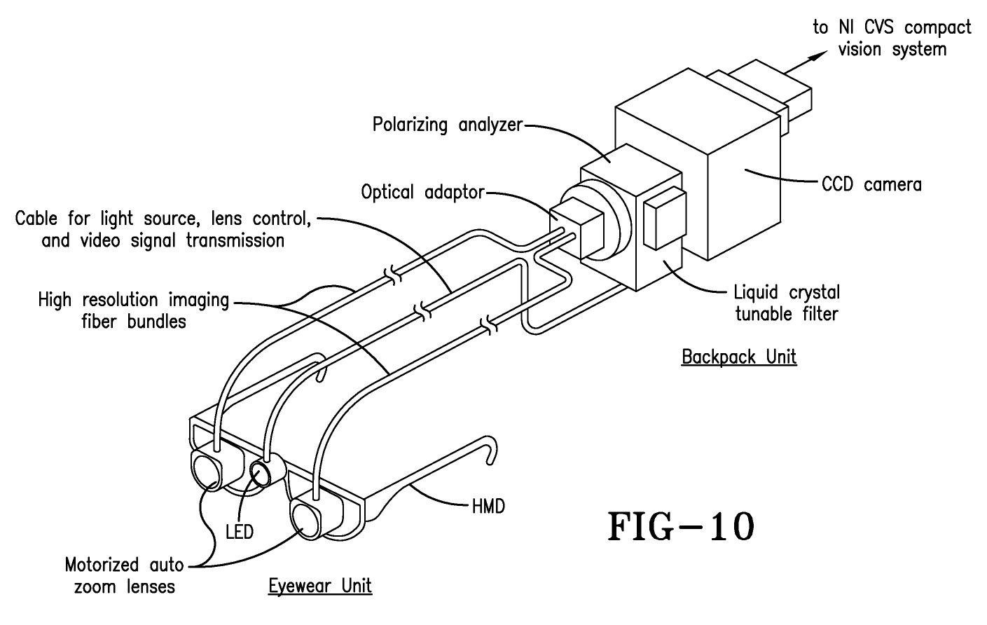

An alternative wearable surgical navigation system for intraoperative cancer imaging is illustrated in FIG. 10. As shown in FIG. 10, the wearable system includes special eyewear and a control unit. Optionally, the eyewear unit may be based on commercially available head mounted display (HMD) systems such as Headplay™ (Headplay Co., Santa Monica, Calif.). Preferably, excitation light may be provided by a Luxeon™ superbright LED (Quadica Developments Inc., Brantford, Ontario) which may be modulated and collimated for pulsed illumination on the surgical site.

Fluorescence emission from tumor-binding ICG-ATT may be collected by motorized zoom lenses (Pentax Imaging, Golden, Colo.) and delivered through high resolution imaging fiber guides to the control unit. The control unit may house the image acquisition and processing module components of the system. The control unit may optionally be housed in a portable backpack unit which may be worn by the surgeon. If so outfitted, such a backpack unit would be a compact image acquisition and processing module that would allow unlimited and unrestricted mobility to the surgeon during any given breast cancer surgery case.

The unit shown in FIG. 10 includes an optical adaptor, a filter (e.g., a VariSpec™ liquid tunable filter (Cri, Woburn, Mass.)), and a CCD camera. To minimize the weight of the backpack unit, a single CCD camera with custom designed view separation optics similar to that in color photography may be used for simultaneous acquisition of background and fluorescence images from two fiber bundles. Friedman, History of Color Photography, 2 ed: Focal P (1968). The above components (i.e.: LED, motorized zoom lenses, HMD, liquid crystal tunable filter, and CCD camera) are all connected to a National Instrument CVS compact vision system with embedded programs for real time control and synchronization of LED flashing, lens zooming, image acquisition and HMD display. A remote computer may communicate with the compact vision system through Ethernet connection for continuous monitoring and control of the eyewear unit and the backpack unit.

During surgery, the motorized zoom lenses may adaptively adjust their focal points as the surgeon changes his posture. As the LED illuminates, excitation images (background) are taken at the video speed. Whereas, emission images (fluorescence) are captured at specific wavelengths and calculated for antibody-fluorophore concentration. The fused images (background+fluorescence) are then sent to HMD for visual display. With the view separation technique, video rate of 30 fps for the background image with shorter than 200 msec update rate for fluorescence image fusion are achieved.

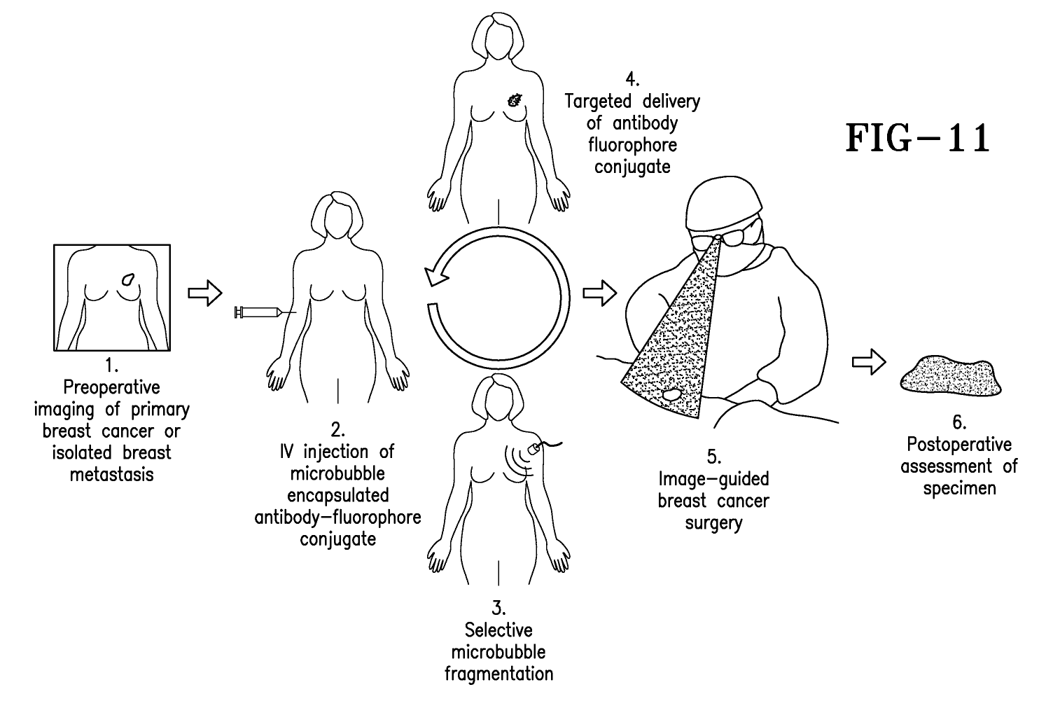

In practice, the clinical application of this cancer surgical process may comprise the following steps, as illustrated in FIG. 11: (1) The primary tumor and/or isolated metastases may be identified in conjunction with preoperative imaging, such as PET/CT or MRI; (2) The microbubble encapsulated antibody-fluorophore conjugate may be administered through IV injection; (3) An ultrasound pulse may be applied on the local tissue to selectively destroy microbubbles; (4) The cancer-specific antibody-fluorophore conjugate may be released from the microbubble carrier and penetrate through the vessel wall to accumulates in the surgical site; (5) After several cycles of microbubble injection and fragmentation followed by a period of washing-out, tumor resection may then be carried out with the image-guidance by the proposed goggle system; and (6) Resected tumor specimens may be assessed at the time of resection by micro PET/CT or by a portable fluorescence system to verify the removal of the primary tumor, isolated metastases, and occult disease and to confirm the adequacy of the resection margins.

### EXAMPLE

### Imaging Results in Small Animal Model

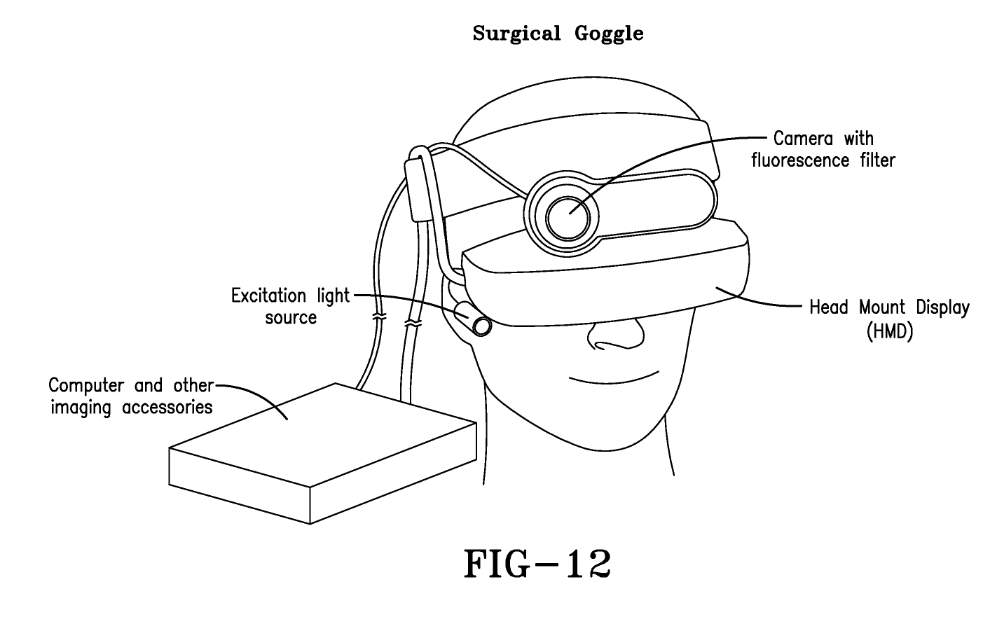

A simplified goggle prototype is shown in FIG. 12. An exemplary goggle system may comprise a camera with fluorescence filter, a multi-wavelength excitation light source, a head mount display (HMD), a laptop computer, and other imaging processing accessories, as shown in FIG. 12. A Labview program may be used to fuse the background image and the fluorescence emission captured by the camera, and project to HMD for real-time image guidance.

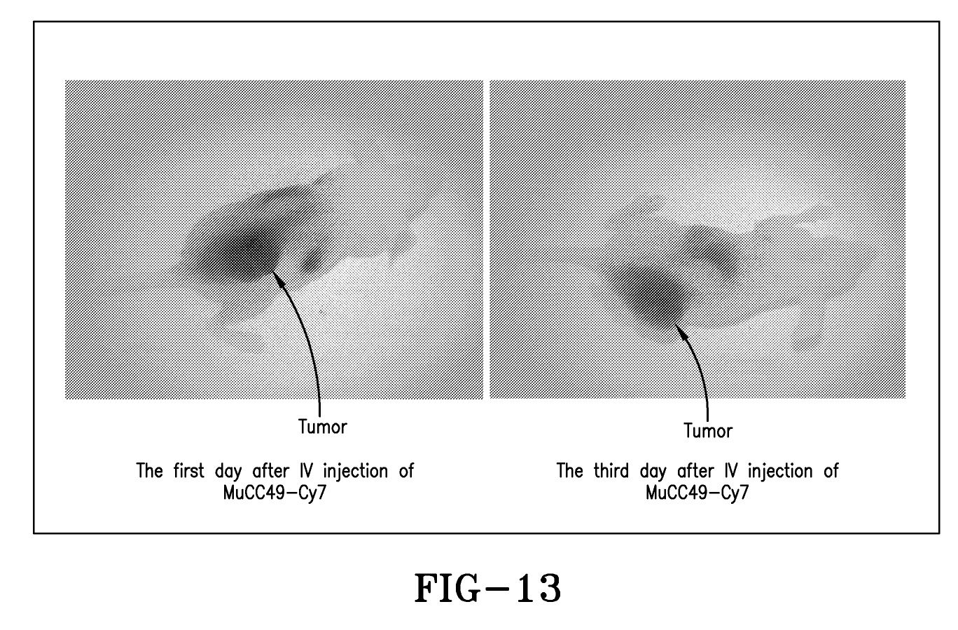

As demonstrated in FIG. 13, useful fluorescence images can be visualized after an IV injection of MuCC49-Cy7 conjugate on a LS174T colon cancer xenograft nude mouse. The fluorescence imaging system used to obtain the images shown in FIG. 13 is similar to the exemplary system described in FIG. 12, except that the images were projected to a computer monitor instead of a HMD. However, the core technique is similar to that of the system described in FIG. 12.

As is evident from FIG. 13, tumor can be readily identified from the images. Non cancer-specific deposition of MuCC49-Cy7 is observed in the first day of the IV injection. However, non-specific deposition is gradually diminished. In this experiment, the maximal tumor-to-normal tissue ratio and the optimal image contrast may be obtained a week after the IV injection of MuCC49-Cy7 conjugate. Because the fluorescence agent has an emission peak in the near infrared range, the goggle system should be able to see tissues as deep as 5 cm (see FIG. 12). The lateral resolution of the goggle system depends on the camera resolution and the area of interest. With the conventional CCD camera and typical ROI of 5 cm×5 cm, at least 50 μm lateral resolution should be achievable.

In an exemplary embodiment, the goggle system may have 3D imaging capability simulating that of human eyes. In this embodiment, more than two cameras are used. In addition, several techniques may be used to extend the 3D imaging capability, either alone or in combination. In one embodiment, a linear polarizer placed in front of the excitation LED and a polarizing analyzer placed in front of the camera are able to decouple the epithelial and stromal fluorescence for maximal contrast between malignant and normal tissue boundaries. In another embodiment, a Digital Micromirror Device (DMD) may be employed to modulate the excitation light with different spatial frequencies for depth-resolved fluorescence imaging. The fundamental solution for 3D tumor tomography is multimodal imaging that combines structural and functional modalities. Photoacoustic tomography is one of such modalities which may be employed.

To enhance the tumor/background ratio at the surgical site and to reduce the systemic concentration of the imaging agent, the tumor-specific antibody-fluorophore conjugate may be delivered through a microbubble carrier. Microbubble is an FDA approved contrast agent originally used as a red blood cell tracer for clinical assessment of heart function. It has also been used to enhance the contrast of power Doppler ultrasound in breast cancer diagnosis. Kettenbach et al., Eur J Radiol, vol. 53, pp. 238-44 (2005). Microbubbles have recently been used in target cancer imaging and target drug delivery. Tsuitsui et al., Cardiovascular Ultrasound, vol. 2, 13 (2004); Villanueva et al., Cardiol Clin, vol. 22, pp. 283-98, vii (2004); Unger et al., Adv Drug Deliv Rev, vol. 56, pp. 1291-314 (2004).

The use of Microbubble agents permits the following two consecutive strategies for target delivery of antibody-fluorophore conjugates: first, microbubbles may be delivered and accumulated in the tumor vasculature by several mechanisms such as targeting increased expression of VEGF receptors, adhesion to endothelium through accumulated leukocytes, and targeting increased vascular permeability (Villanueva et al., Cardiol Clin, vol. 22, pp. 283-98, vii (2004); Foster, VisualSonics Satellite Symposium (2006); McDonald et al., Cancer Res, vol. 62, pp. 5381-5 (2002)); second, microbubbles may be selectively destroyed at the surgical site so that the imaging agents are released, penetrated through endothelium, and target the cancer tissue. Clinical ultrasound may be used for selective fragmentation of microbubbles due to the low pressure threshold for ultrasound induced fragmentation. Chomas et al., J. Biomed. Opt., vol. 6, pp. 141-150 (2001). Previous studies have demonstrated that microbubbles with the size of around 1 μm can be effectively destroyed at clinically useful ultrasound frequencies such as 1 MHz. Unger et al., Adv Drug Deliv Rev, vol. 56, pp. 1291-314 (2004).

In specific embodiment, nanobubbles may be employed to deliver the tumor-specific antibody-fluorophore conjugate. In recent years, many research works have been focused on drug-loaded PLGA nanobubbles and nanoparticles for targeted cancer imaging and therapy. Unlike microbubbles, nanobubbles may directly penetrate through leaky tumor vasculature to interstitial tissue spaces. Typically, nanobubbles and microbubbles contain gaseous cores of low permeability, such as perfluorocarbon (PFC). Liquid PFC of low boiling point was also encapsulated in nanobubbles for ultrasound-induced phase shift at the patient's body temperature. In addition to ultrasound applications, gas-filled microbubbles have also been used to enhance the scattering contrast for optical coherence tomography and DOT. However, other than enhancing scattering contrast, no further imaging application is reported for microbubbles and nanobubbles.

By conjugating nanobubbles (or microbubbles) with a cancer-specific ligand, and by encapsulating fluorescence contrast agents (such as Cy7) and anti-cancer drugs (such as doxorubicin) in nanobubbles, specific embodiments improve the clinical efficiency and safety of anti-cancer drugs by optimizing the dosage, increasing the local deposition, and reducing the systemic distribution.

Although the exemplary embodiments may be directed at markers associated with neoplastic tissue, the systems and methods described herein are broadly applicable for other target tissues, medical conditions, therapies, diagnostics, and/or research purposes. As an illustrative example, using materials and methods described herein, a fluorescent targeting agent directed at markers associated with atherosclerotic plaques may be generated. For example, a monoclonal antibodies with demonstrated specificity to an atherosclerotic plaques (e.g., the targeting agents described in U.S. Pat. No. 6,025,477, incorporated by reference in its entirety) may be operably linked to a fluorophore of an exemplary embodiment above. Using exemplary fluorescent detection systems (e.g., a fluorescence microscopic endoscope) and methods described above, a patient's condition may be visually diagnosed, and furthermore, therapeutic agents and or other treatments may be delivered intra-operatively or otherwise. Other applications within the scope of the present invention include plastic surgery where functional and molecular characteristics of the tissue flap can be visualized to ensure successful transplantation and caridovascular surgery where mycardium ischemia can be visualized in real-time for treatment guidance.

### PUBLICATIONS

The following references and others cited herein but not listed here, to the extent that they provide exemplary procedural and other details supplementary to those set forth herein, are specifically incorporated herein by reference.
Breast Cancer Facts & FIGURES 2005-2006. In. Atlanta: American Cancer Society, Inc.
Breast Cancer Facts & FIGURES 2007-2008. In. Atlanta: American Cancer Society, Inc.
Endocrinology of the breast: basic and clinical aspects. Turin, Italy, Sep. 19-22, 1984. Ann N Y Acad Sci 1986, 464:1-640.
Mammography Quality Standards Act Regulations [http://www.fda.gov/cdrh/mammography/frmamcom2.html#s90012]
RIGScan CR: RIGScan CR49. Drugs R D 2004, 5(4):240-241.
Agnese D M, Abdessalam S F, Burak W E, Jr., Arnold M W, Soble D, Hinkle G H, Young D, Khazaeli M B, Martin E W, Jr.: Pilot study using a humanized CC49 monoclonal antibody (HuCC49DeltaCH2) to localize recurrent colorectal carcinoma. Ann Surg Oncol 2004, 11(2):197-202.
Aitken D R, Hinkle G H, Thurston M O, Tuttle S E, Martin D T, Olsen J, Haagensen D E, Jr., Houchens D, Martin E W, Jr.: A gamma-detecting probe for radioimmune detection of CEA-producing tumors. Successful experimental use and clinical case report. Dis Colon Rectum 1984, 27(5):279-282.
Ammi A, Cleveland R, Mamou J, Wang G, Bridal L, O'Brien W: Ultrasonic contrast agent shell rupture detected by internal cavitation and rebound signals. IEEE Trans Ultrason Ferroelectr Freq Control 2006, 53(1):126-135.
Arnold M W, Hitchcock C L, Young D C, Burak W E, Jr., Bertsch D J, Martin E W, Jr.: Intra-abdominal patterns of disease dissemination in colorectal cancer identified using radioimmunoguided surgery. Dis Colon Rectum 1996, 39(5):509-513.
Arnold M W, Schneebaum S, Berens A, Mojzisik C, Hinkle G, Martin E W, Jr.: Radioimmunoguided surgery challenges traditional decision making in patients with primary colorectal cancer. Surgery 1992, 112(4):624-629; discussion 629-630.
Arnold M W, Schneebaum S, Berens A, Petty L, Mojzisik C, Hinkle G, Martin E W, Jr.: Intraoperative detection of colorectal cancer with radioimmunoguided surgery and CC49, a second-generation monoclonal antibody. Ann Surg 1992, 216(6):627-632.
Arnold M W, Schneebaum S, Martin E W, Jr.: Radioimmunoguided Surgery in the Treatment and Evaluation of Rectal Cancer Patients. Cancer Control 1996, 3(1):42-45.
Arnold M W, Young D C, Hitchcock C L, Schneebaum S, Martin E W, Jr.: Radioimmunoguided surgery in primary colorectal carcinoma: an intraoperative prognostic tool and adjuvant to traditional staging. Am J Surg 1995, 170(4):315-318.
Arnold M W, Young D M, Hitchcock C L, Barbera-Guillem E, Nieroda C, Martin E W, Jr.: Staging of colorectal cancer: biology vs. morphology. Dis Colon Rectum 1998, 41(12):1482-1487.
Aronson R: Boundary conditions for diffusion of light. JOSA A 1995, 12(11):2532.
Arridge S R, Dehghani H, Schweiger M, Okada E: The finite element model for the propagation of light in scattering media: a direct method for domains with nonscattering regions. Med Phys 2000, 27(1):252-264.
Assenheimer M, Layer-Moskovitz O, Malonek D, Manor D, Nahaliel U, Nitzan R, Saad A: The T-SCAN technology: electrical impedance as a diagnostic tool for breast cancer detection. Physiol Meas 2001, 22(1):1-8.
Bankman I: Handbook of Medical Imaging: Processing and Analysis: Academic Press; 2000.
Bell D S, Bamber J C, Eckersley R J: Segmentation and analysis of colour Doppler images of tumour vasculature. Ultrasound Med Biol 1995, 21(5):635-647.
Bercoff J, Chaffai S, Tanter M, Sandrin L, Catheline S, Fink M, Gennisson J L, Meunier M: In vivo breast tumor detection using transient elastography. Ultrasound Med Biol 2003, 29(10):1387-1396.
Bertsch D J, Burak W E, Jr., Young D C, Arnold M W, Martin E W, Jr.: Radioimmunoguided Surgery system improves survival for patients with recurrent colorectal cancer. Surgery 1995, 118(4):634-638; discussion 638-639.
Bertsch D J, Burak W E, Jr., Young D C, Arnold M W, Martin E W, Jr.: Radioimmunoguided surgery for colorectal cancer. Ann Surg Oncol 1996, 3(3):310-316.
Boas D A: A fundamental limitation of linearized algorithms for diffuse optical tomography. Optics Express 1997, 1(13):404-413.
Boas D A, Culver J P, Stott J J, Dunn A K: Three dimensional Monte Carlo code for photon migration through complex heterogeneous media including the adult human head. Optics Express 2002, 10(3):159-170.
Bohren C F, Huffman D R: Absorption and scattering of light by small particles. New York: Wiley; 1983.
Burak W E, Jr., Agnese D M, Povoski S P: Advances in the surgical management of early stage invasive breast cancer. Curr Probl Surg 2004, 41(11):882-935.
Burt B M, Humm J L, Kooby D A, Squire O D, Mastorides S, Larson S M, Fong Y: Using positron emission tomography with [(18)F]FDG to predict tumor behavior in experimental colorectal cancer. Neoplasia 2001, 3(3):189-195.
Carson P L, Fowlkes J B, Roubidoux M A, Moskalik A P, Govil A, Normolle D, LeCarpentier G, Nattakom S, Helvie M, Rubin J M: 3-D color Doppler image quantification of breast masses. Ultrasound Med Biol 1998, 24(7):945-952.
Carson P L, Giger M: Biomedical Imaging Research Opportunities Workshop: report and recommendations overview of the workshop. Acad Radiol 2003, 10(8):882-886.
Chance B, Nioka S, Zhang J, Conant E F, Hwang E, Briest S, Orel S G, Schnall M D, Czerniecki B J: Breast cancer detection based on incremental biochemical and physiological properties of breast cancers: a six-year, two-site study. Acad Radiol 2005, 12(8):925-933.
Chang R F, Wu W J, Moon W K, Chen W M, Lee W, Chen DR: Segmentation of breast tumor in three-dimensional ultrasound images using three-dimensional discrete active contour model. Ultrasound Med Biol 2003, 29(11):1571-1581.
Chen W M, Chang R F, Moon W K, Chen DR: Breast cancer diagnosis using three-dimensional ultrasound and pixel relation analysis. Ultrasound Med Biol 2003, 29(7):1027-1035.
Cheng X, Mao J M, Xu X, Elmandjra M, Bush R, Christenson L, O'Keefe B, Bry J: Post-occlusive reactive hyperemia in patients with peripheral vascular disease. Clin Hemorheol Microcirc 2004, 31(1):11-21.
Cheng X, Mao J-m, Xu X, Elmandjra M, Bush R, Christenson L, O'keefe B, Bry J: Post-occlusive reactive hyperemia in patients with peripheral vascular disease. Clinical Hemorheology and Microcirculation 2004, 31:11-21.
Cheng X, Xu X: Study of the pressure effect in near infrared spectroscopy. Proc SPIE 2003, 4955:397-406.
Cheng X, Xu X, Zhou S, Wang L, Hu B, Li F, Wang M, Zhou C, Li H, Zhang H: A novel optical scanning system for breast cancer imaging. Proc SPIE 2001, 4244:468-473.
Chomas J E, Dayton P, May D, Ferrara K: Threshold of fragmentation for ultrasonic contrast agents. J Biomed Opt 2001, 6(2):141-150.
Cohen A M, Martin E W, Jr., Layery I, Daly J, Sardi A, Aitken D, Bland K, Mojzisik C, Hinkle G: Radioimmunoguided surgery using iodine 125 B72.3 in patients with colorectal cancer. Arch Surg 1991, 126(3):349-352.
Colcher D, Esteban J M, Carrasquillo J A, Sugarbaker P, Reynolds J C, Bryant G, Larson S M, Schlom J: Quantitative analyses of selective radiolabeled monoclonal antibody localization in metastatic lesions of colorectal cancer patients. Cancer Res 1987, 47(4):1185-1189.
Colcher D, Hand P H, Nuti M, Schlom J: Differential binding to human mammary and nonmammary tumors of monoclonal antibodies reactive with carcinoembryonic antigen. Cancer investigation 1983, 1(2):127-138.
Colcher D, Hand P H, Wunderlich D, Nuti M, Teramoto Y A, Kufe D, Schlom J: Monoclonal antibodies to human mammary carcinoma associated antigens and their potential uses for diagnosis, prognosis, and therapy. Laboratory and Research Methods in Biology and Medicine 1983, 8(Immunodiagnostics):215-258.
Colcher D, Keenan A M, Larson S M, Schlom J: Prolonged binding of a radiolabeled monoclonal antibody (B72.3) used for the in situ radioimmunodetection of human colon carcinoma xenografts. Cancer Res 1984, 44(12 Pt 1):5744-5751.
Colcher D, Milenic D, Roselli M, Raubitschek A, Yarranton G, King D, Adair J, Whittle N, Bodmer M, Schlom J: Characterization and biodistribution of recombinant and recombinant/chimeric constructs of monoclonal antibody B72.3. Cancer Res 1989, 49(7):1738-1745.
Colcher D, Minelli M F, Roselli M, Muraro R, Simpson-Milenic D, Schlom J: Radioimmunolocalization of human carcinoma xenografts with B72.3 second generation monoclonal antibodies. Cancer Res 1988, 48(16):4597-4603.
Colcher D, Zalutsky M, Kaplan W, Kufe D, Austin F, Schlom J: Radiolocalization of human mammary tumors in athymic mice by a monoclonal antibody. Cancer research 1983, 43(2):736-742.
Cote R J, Houchens D P, Hitchcock C L, Saad A D, Nines R G, Greenson J K, Schneebaum S, Arnold M W, Martin E W, Jr.: Intraoperative detection of occult colon cancer micrometastases using 125 I-radiolabled monoclonal antibody CC49. Cancer 1996, 77(4):613-620.
Cuccia D J, Bevilacqua F, Durkin A J, Tromberg B J: Modulated imaging: quantitative analysis and tomography of turbid media in the spatial-frequency domain. Opt Lett 2005, 30(11):1354-1356.
Debnath J, Muthuswamy S K, Brugge J S: Morphogenesis and oncogenesis of MCF-10A mammary epithelial acini grown in three-dimensional basement membrane cultures. Methods 2003, 30(3):256-268.
Detry R J, Kartheuser A H, Lagneaux G, Rahier J: Preoperative lymph node staging in rectal cancer: a difficult challenge. Int J Colorectal Dis 1996, 11(5):217-221.
Diebold T, Jacobi V, Scholz B, Hensel C, Solbach C, Kaufmann M, Viana F, Balzer J, Peters J, Vogl T: Value of electrical impedance scanning (EIS) in the evaluation of BI-RADS III/IV/V-lesions. Technol Cancer Res Treat 2005, 4(1):93-97.
Domingo R J, Reilly R M: Pre-targeted radioimmunotherapy of human colon cancer xenografts in athymic mice using streptavidin-CC49 monoclonal antibody and 90Y-DOTA-biotin. Nucl Med Commun 2000, 21(11):89-96.
Durduran T, Choe R, Yu G, Zhou C, Tchou J C, Czerniecki B J, Yodh A G: Diffuse optical measurement of blood flow in breast tumors. Opt Lett 2005, 30(21):2915-2917.
Ewers R, Schicho K, Undt G, Wanschitz F, Truppe M, Seemann R, Wagner A: Basic research and 12 years of clinical experience in computer-assisted navigation technology: a review. Int J Oral Maxillofac Surg 2005, 34(1):1-8.
Fang L, Holford N H, Hinkle G, Cao X, Xiao J J, Bloomston M, Gibbs S, Saif O H, Dalton J T, Chan K K et al: Population pharmacokinetics of humanized monoclonal antibody HuCC49deltaCH2 and murine antibody CC49 in colorectal cancer patients. J Clin Pharmacol 2007, 47(2):227-237.
Fang L, Zhong Y, Yang M, Chan K K, Jr E T M, Sun D: In Vivo Fluorescent Imaging for Antibody-Directed Enzyme Prodrug Therapy (ADEPT) and Tumor Detection Using HuCC49H2-galactosidase Conjugate. Clin Cancer Res 2007, Submitted.
Fantini S, Heffer E, Siebold H, Schutz O: Using near-infrared light to detect breast cancer. Optics & Photonics News 2003 (November):24-29.
Farrell T J, Patterson M S, Wilson B: A diffusion theory model of spatially resolved, steady-state diffuse reflectance for the noninvasive determination of tissue optical properties in vivo. Medical Physics 1992, 19(4):879-888.
Ferrari M, Mottola L, Quaresima V: Principles, techniques, and limitations of near infrared spectroscopy. Can J Appl Physiol 2004, 29(4):463-487.
Functional and molecular micro-ultrasound imaging with microbubble contrast agents [http://www.visualsonics.com/imaging/flash/foster_washington/]
Frangioni J V: In vivo near-infrared fluorescence imaging. Curr Opin Chem Biol 2003, 7(5):626-634.
Freitas S, Merkle H P, Gander B: Microencapsulation by solvent extraction/evaporation: reviewing the state of the art of microsphere preparation process technology. J Control Release 2005, 102(2):313-332.
Friedman J S: History of Color Photography, 2 edn: Focal P; 1968.
Fuller S C, Strong E B: Computer applications in facial plastic and reconstructive surgery. Curr Opin Otolaryngol Head Neck Surg 2007, 15(4):233-237.
Gabriel C, Gabriel S: Compilation of the dielectric properties of body tissues at RF and microwave frequencies. In: Final Report AL/OE-1996-0037. AFOSR/NL Boiling AFB; 1996.
Gallagher B M, Ansari A, Atkins H, Casella V, Christman D R, Fowler J S, Ido T, MacGregor R R, Som P, Wan C N et al: Radiopharmaceuticals XXVII. 18F-labeled 2-deoxy-2-fluoro-d-glucose as a radiopharmaceutical for measuring regional myocardial glucose metabolism in vivo: tissue distribution and imaging studies in animals. J Nucl Med 1977, 18(10):990-996.
Gambhir S S: Molecular imaging of cancer with positron emission tomography. Nat Rev Cancer 2002, 2(9):683-693.
Gambhir S S, Czernin J, Schwimmer J, Silverman D H, Coleman R E, Phelps M E: A tabulated summary of the FDG PET literature. J Nucl Med 2001, 42(5 Suppl):1S-93S.
Gardner B: Five-year survival after extended resection of colon cancer. J Surg Oncol 1987, 34(4):258-261.
Gaudette R: Constrained reconstruction techniques for diffuse optical tomography, Ph.D. thesis. PhD. Tufts University; 2000.
Gautherie M: Thermobiological assessment of benign and malignant breast diseases. Am J Obstet Gynecol 1983, 147(8):861-869.
Gemert M v, Welch A J, Star W M: One-dimensional Transport Theory. In: Optical-thermal response of laser—irradiated tissue. Edited by Welch A J, van Gemert M J C. New York: Plenum; 1995: 47-73.
Ghosh N, Majumder S K, Patel H S, Gupta P K: Depth-resolved fluorescence measurement in a layered turbid medium by polarized fluorescence spectroscopy. Opt Lett 2005, 30(2):162-164.
Graves S S, Dearstyne E, Lin Y, Zuo Y, Sanderson J, Schultz J, Pantalias A, Gray D, Axworthy D, Jones H M et al: Combination therapy with Pretarget CC49 radioimmunotherapy and gemcitabine prolongs tumor doubling time in a murine xenograft model of colon cancer more effectively than either monotherapy. Clin Cancer Res 2003, 9(10 Pt 1):3712-3721.
Greiner J W, Guadagni F, Roselli M, Ullmann C D, Nieroda C, Schlom J: Improved experimental radioimmunotherapy of colon xenografts by combining 131I-CC49 and interferon-gamma. Dis Colon Rectum 1994, 37(2 Suppl):S100-105.
Greiner J W, Ullmann C D, Nieroda C, Qi C F, Eggensperger D, Shimada S, Steinberg S M, Schlom J: Improved radioimmunotherapeutic efficacy of an anticarcinoma monoclonal antibody (131I-CC49) when given in combination with gamma-interferon. Cancer Res 1993, 53(3):600-608.
Gu Y, Bourke V A, Kim J G, Constantinescu A, Mason R P, Liu H: Dynamic response of breast tumor oxygenation to hyperoxic respiratory challenge monitored with three oxygen-sensitive parameters. Appl Opt 2003, 42(16):2960-2967.
Gurfinkel M, Ke S, Wen X, Li C, Sevick-Muraca EM: Near-infrared fluorescence optical imaging and tomography. Dis Markers 2003, 19(2-3):107-121.
Haberkorn U, Morr I, Oberdorfer F, Bellemann M E, Blatter J, Altmann A, Kahn B, van Kaick G: Fluorodeoxyglucose uptake in vitro: aspects of method and effects of treatment with gemcitabine. J Nucl Med 1994, 35(11):1842-1850.
Haberkorn U, Ziegler S I, Oberdorfer F, Trojan H, Haag D, Peschke P, Berger M R, Altmann A, van Kaick G: FDG uptake, tumor proliferation and expression of glycolysis associated genes in animal tumor models. Nucl Med Biol 1994, 21(6):827-834.
Hall T J, Zhu Y, Spalding C S: In vivo real-time freehand palpation imaging. Ultrasound Med Biol 2003, 29(3):427-435.
Hansford D J, Ferrari M: Micromachined interfaces: new approaches in cell immunoisolation and biomolecular separation. Biomolecular Engineering 2000, 17(1):23-36.
Heremans J P, Wang S S, Schroeder T, L. L. Nagy: Diesel Engine Lubricating Oil Contaminant Sensor Method. In. USA; 2005.
Herve L, Koenig A, Da Silva A, Berger M, Boutet J, Dinten J M, Peltie P, Rizo P: Noncontact fluorescence diffuse optical tomography of heterogeneous media. Appl Opt 2007, 46(22):4896-4906.
Hirata T, Kogiso H, Morimoto K, Miyamoto S, Taue H, Sano S, Muguruma N, Ito S, Nagao Y: Synthesis and reactivities of 3-indocyanine-green-acyl-1,3-thiazolidine-2-thione (ICG-ATT) as a new near-infrared fluorescent-labeling reagent. Bioorg Med Chem 1998, 6(11):2179-2184.
Horan Hand P, Colcher D, Salomon D, Ridge J, Noguchi P, Schlom J: Influence of spatial configuration of carcinoma cell populations on the expression of a tumor-associated glycoprotein. Cancer Res 1985, 45(2):833-840.
Ibanez L, Schroeder W, Ng L, Cates J: The ITK Software. Kitware Inc 2004.
Jain R A: The manufacturing techniques of various drug loaded biodegradable poly(lactide-co-glycolide) (PLGA) devices. Biomaterials 2000, 21(23):2475-2490.
Jakubowski D B, Cerussi A E, Bevilacqua F, Shah N, Hsiang D, Butler J, Tromberg B J: Monitoring neoadjuvant chemotherapy in breast cancer using quantitative diffuse optical spectroscopy: a case study. J Biomed Opt 2004, 9(1):230-238.
James E. Chomas P D, Donovan May, and Kathy Ferrara: Threshold of fragmentation for ultrasonic contrast agents. J Biomed Opt 2001, 6(2):141-150.
Jemal A, Siegel R, Ward E, Murray T, Xu J, Thun M J: Cancer statistics, 2007. C A Cancer J Clin 2007, 57(1):43-66.
Jiang S, Pogue B W, Paulsen K D, Kogel C, Poplack S: In vivo near-infrared spectral detection of pressure-induced changes in breast tissue. Optics Letters 2003, 28(14):1212-1214.
Johnson V G, Schlom J, Paterson A J, Bennett J, Magnani J L, Colcher D: Analysis of a human tumor-associated glycoprotein (TAG-72) identified by monoclonal antibody B72.3. Cancer Res 1986, 46(2):850-857.
Jossinet J: Variability of impedivity in normal and pathological breast tissue. Med Biol Eng Comput 1996, 34(5):346-350.
Jossinet J: The impedivity of freshly excised human breast tissue. Physiol Meas 1998, 19(1):61-75.
Kepshire D S, Davis S C, Dehghani H, Paulsen K D, Pogue B W: Subsurface diffuse optical tomography can localize absorber and fluorescent objects but recovered image sensitivity is nonlinear with depth. Appl Opt 2007, 46(10):1669-1678.
Kerner T E, Paulsen K D, Hartov A, Soho S K, Poplack S P: Electrical impedance spectroscopy of the breast: clinical imaging results in 26 subjects. IEEE Trans Med Imaging 2002, 21(6):638-645.
Keshtkar A, Smallwood R H: Electrical impedance spectroscopy and the diagnosis of bladder pathology. Physiol Meas 2006, 27(7):585-596.
Kettenbach J, Helbich T H, Huber S, Zuna I, Dock W: Computer-assisted quantitative assessment of power Doppler US: effects of microbubble contrast agent in the differentiation of breast tumors. Eur J Radiol 2005, 53(2):238-244.
Kim J A, Triozzi P L, Martin E W, Jr.: Radioimmunoguided surgery for colorectal cancer. Oncology (Williston Park) 1993, 7(2):55-60; discussion 60, 63-54.
Kim J B: Three-dimensional tissue culture models in cancer biology. Semin Cancer Biol 2005, 15(5):365-377.
Korpanty G, Grayburn P A, Shohet R V, Brekken R A: Targeting vascular endothelium with avidin microbubbles. Ultrasound Med Biol 2005, 31(9):1279-1283.
Kubota K: From tumor biology to clinical Pet: a review of positron emission tomography (PET) in oncology. Ann Nucl Med 2001, 15(6):471-486.
Kuppusamy P, Li H, Ilangovan G, Cardounel A J, Zweier J L, Yamada K, Krishna M C, Mitchell J B: Noninvasive imaging of tumor redox status and its modification by tissue glutathione levels. Cancer Res 2002, 62(1):307-312.
LaValle G J, Martinez D A, Sobel D, DeYoung B, Martin E W, Jr.: Assessment of disseminated pancreatic cancer: a comparison of traditional exploratory laparotomy and radioimmunoguided surgery. Surgery 1997, 122(5):867-871; discussion 871-863.
Li A, Miller E L, Kilmer M E, Brukilacchio T J, Chaves T, Stott J, Zhang Q, Wu T, Chorlton M, Moore R H et al: Tomographic optical breast imaging guided by three-dimensional mammography. Appl Opt 2003, 42(25):5181-5190.
Lin Y, Gao H, Nalcioglu O, Gulsen G: Fluorescence diffuse optical tomography with functional and anatomical a priori information: feasibility study. Phys Med Biol 2007, 52(18):5569-5585.
Mahmood U, Tung C H, Bogdanov A, Jr., Weissleder R: Near-infrared optical imaging of protease activity for tumor detection. Radiology 1999, 213(3):866-870.
Mahmood U, Weissleder R: Near-infrared optical imaging of proteases in cancer. Mol Cancer Ther 2003, 2(5):489-496.
Malich A, Boehm T, Facius M, Freesmeyer M, Azhari T, Werner B, Anderson R, Kaiser W A: Electrical impedance scanning of lymph nodes: initial clinical and technical findings. Clin Radiol 2002, 57(7):579-586.
Malich A, Boehm T, Facius M, Freesmeyer M G, Fleck M, Anderson R, Kaiser W A: Differentiation of mammographically suspicious lesions: evaluation of breast ultrasound, MRI mammography and electrical impedance scanning as adjunctive technologies in breast cancer detection. Clin Radiol 2001, 56(4):278-283.
Martin D T, Aitken D, Thurston M, Haagensen D, Hinkle G, Olsen J, Houchens D, Ovejera A, Carey L C, Martin E W, Jr.: Successful experimental use of a self-contained gamma detecting device. Curr Surg 1984, 41(3):193-194.
Martin D T, Hinkle G H, Tuttle S, Olsen J, Nabi H, Houchens D, Thurston M, Martin E W, Jr.: Intraoperative radioimmunodetection of colorectal tumor with a hand-held radiation detector. Am J Surg 1985, 150(6):672-675.
Martin E W, Jr., Hinkle G, Mojzisik C, Thurston M O: Radioimmunoguided surgery: a new intraoperative approach to the detection of tumor. Cancer Treat Res 1990, 51:387-411.
Martin E W, Jr., Mojzisik C M, Hinkle G H, Jr., Sampsel J, Siddiqi M A, Tuttle S E, Sickle-Santanello B, Colcher D, Thurston M O, Bell J G et al: Radioimmunoguided surgery using monoclonal antibody. Am J Surg 1988, 156(5):386-392.
Martin E W, Jr., Thurston M O: The use of monoclonal antibodies (MAbs) and the development of an intraoperative hand-held probe for cancer detection. Cancer Invest 1996, 14(6):560-571.
Martin E W, Jr., Thurston M O: Intraoperative radioimmunodetection. Semin Surg Oncol 1998, 15(4):205-208.
Martinez D A, Barbera-Guillem E, LaValle G J, Martin E W, Jr.: Radioimmunoguided Surgery for Gastrointestinal Malignancies: An Analysis of 14 Years of Clinical Experience. Cancer Control 1997, 4(6):505-516.
Martinez-Monge R, Nag S, Nieroda C A, Martin E W: Iodine-125 brachytherapy in the treatment of colorectal adenocarcinoma metastatic to the liver. Cancer 1999, 85(6):1218-1225.
Max Born E W: Principle of Optics, 17 edn: Cambridge University Press; 2002.
McDonald D M, Baluk P: Significance of blood vessel leakiness in cancer. Cancer Res 2002, 62(18):5381-5385.
McKnight A L, Kugel J L, Rossman P J, Manduca A, Hartmann L C, Ehman R L: MR elastography of breast cancer: preliminary results. AJR Am J Roentgenol 2002, 178(6):1411-1417.
Medina R A, Owen G I: Glucose transporters: expression, regulation and cancer. Biol Res 2002, 35(1):9-26.
Meyer H, Garofalakis A, Zacharakis G, Psycharakis S, Mamalaki C, Kioussis D, Economou E N, Ntziachristos V, Ripoll J: Noncontact optical imaging in mice with full angular coverage and automatic surface extraction. Appl Opt 2007, 46(17):3617-3627.
Mincey B A, Perez E A: Advances in screening, diagnosis, and treatment of breast cancer. Mayo Clin Proc 2004, 79(6):810-816.
Mittal S, Black P M: Intraoperative magnetic resonance imaging in neurosurgery: the Brigham concept. Acta Neurochir Suppl 2006, 98:77-86.
Montemurro F, Martincich L, Sarotto I, Bertotto I, Ponzone R, Cellini L, Redana S, Sismondi P, Aglietta M, Regge D: Relationship between DCE-MRI morphological and functional features and histopathological characteristics of breast cancer. Eur Radiol 2007, 17(6):1490-1497.
Morimoto T, Kinouchi Y, Iritani T, Kimura S, Konishi Y, Mitsuyama N, Komaki K, Monden Y: Measurement of the electrical bio-impedance of breast tumors. Eur Surg Res 1990, 22(2):86-92.
Muguruma N, Ito S, Jianxin C, Inoue S, Sano S, Nagao Y, Kido H: Fab fragment labeled with ICG-derivative for detecting digestive tract cancer. Photodiagnosis and Photodynamic Thetapy 2006, 3:177-183.
Mujumdar R B, Ernst L A, Mujumdar S R, Lewis C J, Waggoner A S: Cyanine dye labeling reagents: sulfoindocyanine succinimidyl esters. Bioconjug Chem 1993, 4(2):105-111.
Muraro R, Kuroki M, Wunderlich D, Poole D J, Colcher D, Thor A, Greiner J W, Simpson J F, Molinolo A, Noguchi P et al: Generation and characterization of B72.3 second generation monoclonal antibodies reactive with the tumor-associated glycoprotein 72 antigen. Cancer Res 1988, 48(16):4588-4596.
Nag S, Martinez-Monge R, Mills J, Bauer C, Grecula J, Nieroda C, Martin E: Intraoperative high dose rate brachytherapy in recurrent or metastatic colorectal carcinoma. Ann Surg Oncol 1998, 5(1):16-22.
Nag S, Martinez-Monge R, Nieroda C, Martin E, Jr.: Radioimmunoguided-intraoperative radiation therapy in colorectal carcinoma: a new technique to precisely define the clinical target volume. Int J Radiat Oncol Biol Phys 1999, 44(1):133-137.
Nicholson W B, Lin Y, Oberdier L M, Heremans J P: Method for Measuring High-Frequency Resistance in Diesel Engine Lubrication Oil. In. USA; 2005.
Nieroda C A, Arnold M W, Barbera-Guillem E, Martin E W: [Lymphadenectomy in colorectal carcinoma]. Chirurg 1998, 69(7):717-724.
Nieroda C A, Milenic D E, Carrasquillo J A, Scholm J, Greiner J W: Improved tumor radioimmunodetection using a single-chain Fv and gamma-interferon: potential clinical applications for radioimmunoguided surgery and gamma scanning. Cancer Res 1995, 55(13):2858-2865.
Nieroda C A, Mojzisik C, Hinkle G, Thurston M O, Martin E W, Jr.: Radioimmunoguided surgery (RIGS) in recurrent colorectal cancer. Cancer Detect Prev 1991, 15(3):225-229.
Nieroda C A, Mojzisik C, Sardi A, Farrar W B, Hinkle G, Siddiqi M A, Ferrara P J, James A, Schlom J, Thurston M O et al: Staging of carcinoma of the breast using a hand-held gamma detecting probe and monoclonal antibody B72.3. Surg Gynecol Obstet 1989, 169(1):35-40.
Nieroda C A, Mojzisik C, Sardi A, Ferrara P, Hinkle G, Thurston M O, Martin E W, Jr.: The impact of radioimmunoguided surgery (RIGS) on surgical decision-making in colorectal cancer. Dis Colon Rectum 1989, 32(11):927-932.
Nieroda C A, Mojzisik C, Sardi A, Ferrara P J, Hinkle G, Thurston M O, Martin E W, Jr.: Radioimmunoguided surgery in primary colon cancer. Cancer Detect Prev 1990, 14(6):651-656.
Nioka S D, Kime R D, Sunar U M, Im J M, Izzetoglu M D, Zhang J M, Alacam B M, Chance B D: A novel method to measure muscle blood flow continuously using NIRS kinetics information. Dyn Med 2006, 5(1):5.
Ntziachristos V, Yodh A G, Schnall M, Chance B: Concurrent MRI and diffuse optical tomography of breast after indocyanine green enhancement. Proc Natl Acad Sci USA 2000, 97(6):2767-2772.
Ntziachristos V, Yodh A G, Schnall M D, Chance B: MRI-guided diffuse optical spectroscopy of malignant and benign breast lesions. Neoplasia 2002, 4(4):347-354.
O'Leary M A, Boas D A, Li X D, Chance B, Yodh A G: Fluorescence lifetime imaging in turbid media. Opt Lett 1996(21):158-160.
O'Shaughnessy J: Extending survival with chemotherapy in metastatic breast cancer. Oncologist 2005, 10 Suppl 3:20-29.
Pancino G, Osinaga E, Charpin C, Mistro D, Barque J P, Roseto A: Purification and characterisation of a breast-cancer-associated glycoprotein not expressed in normal breast and identified by monoclonal antibody 83D4. Br J Cancer 1991, 63(3):390-398.
Pavic D, Koomen M A, Kuzmiak C M, Lee Y H, Pisano E D: The role of magnetic resonance imaging in diagnosis and management of breast cancer. Technol Cancer Res Treat 2004, 3(6):527-542.
Peters-Engl C, Frank W, Leodolter S, Medl M: Tumor flow in malignant breast tumors measured by Doppler ultrasound: an independent predictor of survival. Breast Cancer Res Treat 1999, 54(1):65-71.
Peters-Engl C, Medl M, Mirau M, Wanner C, Bilgi S, Sevelda P, Obermair A: Color-coded and spectral Doppler flow in breast carcinomas—relationship with the tumor microvasculature. Breast Cancer Res Treat 1998, 47(1):83-89.
Pogue B W, Jiang S, Dehghani H, Kogel C, Soho S, Srinivasan S, Song X, Tosteson T D, Poplack S P, Paulsen K D: Characterization of hemoglobin, water, and NIR scattering in breast tissue: analysis of intersubject variability and menstrual cycle changes. J Biomed Opt 2004, 9(3):541-552.
Pogue B W, Willscher C, McBride T O, Osterberg U L, Paulsen K D: Contrast-detail analysis for detection and characterization with near-infrared diffuse tomography. Med Phys 2000, 27(12):2693-2700.
Povoski S P, Choudry U H, Dauway E L, Rassekh C H, Ducatman B S: Sentinel lymph node mapping and biopsy for malignant melanoma at a rural-based university medical center. W V Med J 2002, 98(5):194-197.
Povoski S P, Dauway E L, Ducatman B S: Sentinel lymph node mapping and biopsy for breast cancer at a rural-based university medical center: initial experience with intraparenchymal and intradermal injection routes. Breast Cancer 2002, 9(2):134-144.
Povoski S P, Olsen J O, Young D C, Clarke J, Burak W E, Walker M J, Carson W E, Yee L D, Agnese D M, Pozderac R V et al: Prospective randomized clinical trial comparing intradermal, intraparenchymal, and subareolar injection routes for sentinel lymph node mapping and biopsy in breast cancer. Ann Surg Oncol 2006, 13(11):1412-1421.
Povoski S P, Young D C, Walker M J, Carson W E, Yee L D, Agnese D M, Farrar W B: Re-emphasizing the concept of adequacy of intraoperative assessment of the axillary sentinel lymph nodes for identifying nodal positivity during breast cancer surgery. World J Surg Oncol 2007, 5(1):18.
Qiang B, Rana A, Xu R: Reconstruction of tissue heterogeneity by near infrared imaging: a novel algorithm and benchtop validation. Conf Proc IEEE Eng Med Biol Soc 2005, 3:3137-3140.
Raabe A, Krishnan R, Seifert V: Actual aspects of image-guided surgery. Surg Technol Int 2003, 11:314-319.
Rana A, Cao X, Sun D, Xu R: Monitoring Oxygen Dynamics During Pressure Induced Ischemia on Cancer Xenograft Models. IEEE International Conference on Infrared and Millimeter Waves 2006, in print.
Roberson P L, Yokoyama S, Rogers B E, Buchsbaum D J: Three-dimensional dose model for the comparison of 177Lu-HuCC49DeltaCH2 and 177Lu-HuCC49 radioimunotherapy in mice bearing intraperitoneal xenografts. Cancer Biother Radiopharm 2003, 18(2):239-247.
Saidel G M, DiBella J A, Cabrera M E: Metabolic system dynamics: lumped and distributed models. In: Simulations in Biomedicine. Edited by Arnez Z M, vol. V: WIT Press; 2003: 100-110.
Salhab M, Keith L G, Laguens M, Reeves W, Mokbel K: The potential role of dynamic thermal analysis in breast cancer detection. Int Semin Surg Oncol 2006, 3:8.
Sampsel J W, Hinkle G, Nieroda C, Ignaszewski J, Thurston M, Martin E W: Gamma-detecting probe and autoradiographic studies of radiolabeled antibody B72.3 in CX-1 colon xenografts. J Surg Oncol 1990, 45(4):242-249.
Sardi A, Agnone C M, Nieroda C A, Mojzisik C, Hinkle G, Ferrara P, Farrar W B, Bolton J, Thurston M O, Martin E W, Jr.: Radioimmunoguided surgery in recurrent colorectal cancer: the role of carcinoembryonic antigen, computerized tomography, and physical examination. South Med J 1989, 82(10):1235-1244.
Sardi A, Minton J P, Mojzisik C, Nieroda C A, Ferrara P J, Hinkle G H, Thurston M O, Martin E W, Jr.: The use of a hand-held gamma detector improves the safety of isolated limb perfusion. J Surg Oncol 1989, 41(3):172-176.
Sardi A, Minton J P, Nieroda C, Sickle-Santanello B, Young D, Martin E W, Jr.: Multiple reoperations in recurrent colorectal carcinoma. An analysis of morbidity, mortality, and survival. Cancer 1988, 61(9):1913-1919.
Sardi A, Nieroda C A, Siddiqi M A, Minton J P, Martin E W, Jr.: Carcinoembryonic antigen directed multiple surgical procedures for recurrent colon cancer confined to the liver. Am Surg 1990, 56(4):255-259.
Sardi A, Workman M, Mojzisik C, Hinkle G, Nieroda C, Martin E W, Jr.: Intra-abdominal recurrence of colorectal cancer detected by radioimmunoguided surgery (RIGS system). Arch Surg 1989, 124(1):55-59.
Sarvazyan A, Skovoroda A, Emelianov S, JB F, Pipe J, Adler R, Buxton R, Carson P (eds.): Biophysical Bases of Elasticity Imaging. New York: Plenum Press; 1995.
Schmitz C H, Klemer D P, Hardin R, Katz M S, Pei Y, Graber H L, Levin M B, Levina R D, Franco N A, Solomon W B et al: Design and implementation of dynamic near-infrared optical tomographic imaging instrumentation for simultaneous dual-breast measurements. Appl Opt 2005, 44(11):2140-2153.
Schneebaum S, Arnold M W, Houchens D P, Greenson J K, Cote R J, Hitchcock C L, Young D C, Mojzisik C M, Martin E W, Jr.: The significance of intraoperative periportal lymph node metastasis identification in patients with colorectal carcinoma. Cancer 1995, 75(12):2809-2817.
Scholz B, Anderson R: On electical impedance scanning-principles and simulation. Electromedica 2000, 68:35-44.
Schroeder R J, Bostanjoglo M, Rademaker J, Maeurer J, Felix R: Role of power Doppler techniques and ultrasound contrast enhancement in the differential diagnosis of focal breast lesions. Eur Radiol 2003, 13(1):68-79.
Sevick-Muraca E M, Houston J P, Gurfinkel M: Fluorescence-enhanced, near infrared diagnostic imaging with contrast agents. Curr Opin Chem Biol 2002, 6(5):642-650.
Shah N, Cerussi A, Eker C, Espinoza J, Butler J, Fishkin J, Hornung R, Tromberg B: Noninvasive functional optical spectroscopy of human breast tissue. Proc Natl Acad Sci USA 2001, 98(8):4420-4425.
Shah N, Cerussi A E, Jakubowski D, Hsiang D, Butler J, Tromberg B J: The role of diffuse optical spectroscopy in the clinical management of breast cancer. Dis Markers 2003, 19(2-3):95-105.
Shah N, Cerussi A E, Jakubowski D, Hsiang D, Butler J, Tromberg B J: Spatial variations in optical and physiological properties of healthy breast tissue. J Biomed Opt 2004, 9(3):534-540.
Shen J, Cao X, Lee J: Suspension Polymerization of Inverse Emulsion of Water Expandable Polystyrene/Clay Nanocomposites. AiCHE Annual 2005.
Sickle-Santanello B J, O'Dwyer P J, Mojzisik C, Tuttle S E, Hinkle G H, Rousseau M, Schlom J, Colcher D, Thurston M O, Nieroda C et al: Radioimmunoguided surgery using the monoclonal antibody B72.3 in colorectal tumors. Dis Colon Rectum 1987, 30(10):761-764.
Singh B, Smith C W, Hughes R: In vivo dielectric spectrometer. Med Biol Eng Comput 1979, 17(1):45-60.
Skovorda A R, Klishko A N, Gusakian D A, Maevskii E I, Ermilova V D, Oranskaia G A, Sarvazian A P: [Quantitative analysis of mechanical characteristics of pathologically altered soft biological tissues]. Biofizika 1995, 40(6):1335-1340.
Sloka J S, Hollett P D, Mathews M: A quantitative review of the use of FDG-PET in the axillary staging of breast cancer. Med Sci Monit 2007, 13(3):RA37-46.
Spink B C, Cole R W, Katz B H, Gierthy J F, Bradley L M, Spink D C: Inhibition of MCF-7 breast cancer cell proliferation by MCF-10A breast epithelial cells in coculture. Cell Biol Int 2006, 30(3):227-238.
Spink B C, Katz B H, Hussain M M, Pang S, Connor S P, Aldous K M, Gierthy J F, Spink D C: SULT1A1 catalyzes 2-methoxyestradiol sulfonation in MCF-7 breast cancer cells. Carcinogenesis 2000, 21(11):1947-1957.
Stefanadis C, Chrysochoou C, Markou D, Petraki K, Panagiotakos D B, Fasoulakis C, Kyriakidis A, Papadimitriou C, Toutouzas P K: Increased temperature of malignant urinary bladder tumors in vivo: the application of a new method based on a catheter technique. J Clin Oncol 2001, 19(3):676-681.
Stefanadis C, Chrysohoou C, Panagiotakos D B, Passalidou E, Katsi V, Polychronopoulos V, Toutouzas P K: Temperature differences are associated with malignancy on lung lesions: a clinical study. BMC Cancer 2003, 3:1.
Strano S, Gombos E C, Friedland O, Mozes M: Color Doppler imaging of fibroadenomas of the breast with histopathologic correlation. J Clin Ultrasound 2004, 32(7):317-322.
Surowiec A J, Stuchly S S, Barr J B, Swarup A: Dielectric properties of breast carcinoma and the surrounding tissues. IEEE Trans Biomed Eng 1988, 35(4):257-263.
Tadatsu M, Ito S, Muguruma N, Kusaka Y, Inayama K, Bando T, Tadatsu Y, Okamoto K, Ii K, Nagao Y et al: A new infrared fluorescent-labeling agent and labeled antibody for diagnosing microcancers. Bioorg Med Chem 2003, 11(15):3289-3294.
Tadatsu Y, Muguruma N, Ito S, Tadatsu M, Kusaka Y, Okamoto K, Imoto Y, Taue H, Sano S, Nagao Y: Optimal labeling condition of antibodies available for immunofluorescence endoscopy. J Med Invest 2006, 53(1-2):52-60.
Tromberg B J, Shah N, Lanning R, Cerussi A, Espinoza J, Pham T, Svaasand L, Butler J: Non-invasive in vivo characterization of breast tumors using photon migration spectroscopy. Neoplasia 2000, 2(1-2):26-40.
Tsuitsui J, Xue F, Porter R T: The use of microbubbles to target drug delivery. Cardiovascular Ultrasound 2004, 2(23).
Unger E C, Porter T, Culp W, Labell R, Matsunaga T, Zutshi R: Therapeutic applications of lipid-coated microbubbles. Adv Drug Deliv Rev 2004, 56(9):1291-1314.
Utzinger U, Richards-Kortum R R: Fiber optic probes for biomedical optical spectroscopy. J Biomed Opt 2003, 8(1):121-147.
Vaupel P: The role of hypoxia-induced factors in tumor progression. Oncologist 2004, 9 Suppl 5:10-17.
Vaupel P, Harrison L: Tumor hypoxia: causative factors, compensatory mechanisms, and cellular response. Oncologist 2004, 9 Suppl 5:4-9.
Vaupel P, Kallinowski F, Okunieff P: Blood flow, oxygen and nutrient supply, and metabolic microenvironment of human tumors: a review. Cancer Res 1989, 49(23):6449-6465.
Villanueva F S, Wagner W R, Vannan M A, Narula J: Targeted ultrasound imaging using microbubbles. Cardiol Clin 2004, 22(2):283-298, vii.
Vo T V, Hammer P E, Hoimes M L, Nadgir S, Fantini S: Mathematical model for the hemodynamic response to venous occlusion measured with near-infrared spectroscopy in the human forearm. IEEE Trans Biomed Eng 2007, 54(4):573-584.
Wack S, Hajri A, Heisel F, Sowinska M, Berger C, Whelan M, Marescaux J, Aprahamian M: Feasibility, sensitivity, and reliability of laser-induced fluorescence imaging of green fluorescent protein-expressing tumors in vivo. Mol Ther 2003, 7(6):765-773.
Waki A, Fujibayashi Y, Magata Y, Yokoyama A, Sadato N, Tsuchida T, Ishii Y, Yonekura Y: Glucose transporter protein-independent tumor cell accumulation of fluorine-18-AFDG, a lipophilic fluorine-18-FDG analog. J Nucl Med 1998, 39(2):245-250.
Wang L V: Ultrasound-mediated biophotonic imaging: a review of acousto-optical tomography and photo-acoustic tomography. Dis Markers 2003, 19(2-3):123-138.
Warburg O: On the origin of cancer cells. Science 1956, 123(3191):309-314.
Weber G: Enzymology of cancer cells (second of two parts). N Engl J Med 1977, 296(10):541-551.
Weber G: Enzymology of cancer cells (first of two parts). N Engl J Med 1977, 296(9):486-492.
Welch A J, van Gemert M J C, Star W M, Wilson B C: Definitions and overview of tissue optics. In: Optical-Thermal Response of Laser-Irradiated Tissue. Edited by Welch A J, van Germet M J C. New York: Plenum Press; 1995.
Wellman P, Howe R, Dalton E, Kern K A: Breast Tissue Stiffness in Compression is Correlated to Histological Diagnosis. Harvard BioRobotics Laboratory Technical Report 1999.
Xiao J, Horst S, Hinkle G, Cao X, Kocak E, Fang J, Young D, Khazaeli M, Agnese D, Sun D et al: Pharmacokinetics and Clinical Evaluation of (125)I-Radiolabeled Humanized CC49 Monoclonal Antibody (HuCC49DeltaC(H)2) in Recurrent and Metastatic Colorectal Cancer Patients. Cancer Biother Radiopharm 2005, 20(1):16-26.
Xu R, Povoski S, Young D, Mao J: In vivo Dynamic Characterisation of Breast Lesions. World Journal on Surgical Oncology manuscript to be submitted.
Xu R, Qiang B, Mao J: Near infrared imaging of tissue heterogeneity: probe design and sensitivity analysis. Conf Proc IEEE Eng Med Biol Soc 2005, 1:278-281.
Xu R, Qiang B, Mao J, Povoski S: Development of a Handheld Near Infrared Imager for Dynamic Characterization of Biological Tissue in Vivo. Appl Opt, submitted.
Xu R, Qiang B, Olsen J, Povoski S, Yee L, Mao J: Localization and functional parameter reconstruction of suspicious breast lesions by near infrared/ultrasound dual modal imaging. Conf Proc IEEE Eng Med Biol Soc 2005, 5:4473-4476.
Xu R, Rana A: Dynamic Near Infrared Imaging with Ultrasound Guidance (dNIRUS): Analytical Model and Benchtop Validation on Multi-layer Tissue Simulating Phantoms. Proc SPIE 2006, 6086:353-364.
Xu R, Young D, Mao J, Povoski S: A prospective pilot clinical trial evaluating the utility of an in vivo dynamic near infrared system for characterizing suspicious breast lesions. World Journal on Surgical Oncology, submitted.
Xu R X, Povoski S P: Diffuse optical imaging and spectroscopy for cancer. Expert Rev Med Devices 2007, 4(1):83-95.
Xu R X, Povoski S P, Yee L D, Olsen J O, Qiang B, Mao J M: Near Infrared/Ultrasound Dual Modal Imaging for Breast Cancer Detection. Proc SPIE 2006, 6081:44-53.
Xu R X, Qiang B, Mao J J, Povoski S P: Development of a handheld near infrared imager for dynamic characterization of in vivo biological tissue systems. Appl Opt 2007, 46(30):7442-7451.
Xu R X, Qiang B, Olsen J O, Povoski S P, Yee L D, Mao J: Localization and Functional Parameter Reconstruction of Suspicious Breast Lesions by Near Infrared/Ultrasound Dual Modal Imaging. Proceedings of IEEE Engineering in Medicine and Biology Society 2005, 1385:4473-4476.
Xu X, Zhu W, Padival V, Xia M, Cheng X, Bush R, Christenson L, Chan T, Doherty T, Iatridis A: Validation of NIRS In Measuring Tissue Hemoglobin Concentration And Oxygen Saturation Using Benchtop and Isolated Limb Model. Proc SPIE 2003, 4955:369-378.
Yodh A, Chance B: Spectroscopy and Imaging of Tissues with Diffusing Light. Physics Today 1995, 48(3):34-40.
Younes M, Brown R W, Mody D R, Fernandez L, Laucirica R: GLUT1 expression in human breast carcinoma: correlation with known prognostic markers. Anticancer Res 1995, 15(6B):2895-2898.
Younes M, Brown R W, Stephenson M, Gondo M, Cagle P T: Overexpression of Glut1 and Glut3 in stage I nonsmall cell lung carcinoma is associated with poor survival. Cancer 1997, 80(6):1046-1051.
Younes M, Lechago L V, Somoano J R, Mosharaf M, Lechago J: Wide expression of the human erythrocyte glucose transporter Glut1 in human cancers. Cancer Res 1996, 56(5):1164-1167.
Zangheri B, Messa C, Picchio M, Gianolli L, Landoni C, Fazio F: PET/CT and breast cancer. Eur J Nucl Med Mol Imaging 2004, 31 Suppl 1:S135-142.
Zhu Q, Conant E, Chance B: Optical imaging as an adjunct to sonograph in differentiating benign from malignant breast lesions. J Biomed Opt 2000, 5(2):229-236.
Zhu Q, Huang M, Chen N, Zarfos K, Jagjivan B, Kane M, Hedge P, Kurtzman S H: Ultrasound-guided optical tomographic imaging of malignant and benign breast lesions: initial clinical results of 19 cases. Neoplasia 2003, 5(5):379-388.
Zou Y, Guo Z: A review of electrical impedance techniques for breast cancer detection. Med Eng Phys 2003, 25(2):79-90.

Unless otherwise defined, all technical and scientific terms used herein have the same meaning as commonly understood by one of ordinary skill in the art to which this invention pertains. Although methods and materials similar or equivalent to those described herein can be used in the practice or testing of the exemplary embodiments, suitable methods and materials are described above. All publications, patent applications, patents, and other references mentioned herein are incorporated by reference in their entirety. In case of conflict, the present specification, including definitions, will control. In addition, the materials, methods, and examples are illustrative only and not intended to be limiting.

1. A method for intra-operative detection of a neoplastic tissue in an animal, comprising:
(a) administering to an animal an effective amount of a fluorescent targeting agent that specifically binds a marker produced by or associated with the neoplastic tissue;
(b) pausing after the administering step (a) for a period sufficient to allow unbound agent to clear;
(c) after the pausing step (b), locating the neoplastic tissue by optically accessing the animal with a fluorescence detection instrument; and
(d) resecting the neoplastic tissue identified by the fluorescence detection instrument during the accessing step (c).

2. The method of claim 1, further comprising the step of:
identifying a resection margin after the locating step (c).

3. The method of claim 1, wherein the locating step (c) includes the steps of:
penetrating an intervening biological tissue with excitation radiation to excite a fluorophore associated with the targeting agent;
receiving a signal indicating detection of a predetermined concentration of fluorescent emissions.

4. The method of claim 3, wherein:
the fluorescence detection instrument comprises a hand-held probe; and
the locating step (c) further comprises the step of manually positioning the probe adjacent to tissue suspected of being neoplastic.

5. The method of claim 3, wherein:
the fluorescence detection instrument comprises eyewear; and
the receiving step further comprises the step of visualizing an image having information on the position and the relative intensity of the fluorescent emissions within the animal.

6. The method of claim 1, wherein:
the fluorescent targeting agent comprises a purified antibody that binds specifically to TAG-72.

7. The method of claim 1, wherein:
the fluorescent targeting agent comprises a humanized anti-TAG-72 antibody, HuCC49ΔC_{H}2.

8. A cancer surgical method for a human patient, comprising
identifying a tumor using preoperative imaging;
administering an effective amount of a microbubble or nanobubble encapsulated targeting agent through IV injection;
applying an ultrasound pulse on a local tissue to selectively release the targeting agent;
pausing after the applying step for a period sufficient to allow unbound agent to clear;
locating the neoplastic tissue by optically accessing the animal with a fluorescence detection instrument; and
resecting the neoplastic tissue identified by the fluorescence detection instrument during the locating step.

9. The method of claim 8, wherein the locating step includes the steps of:
penetrating an intervening biological tissue with excitation radiation to excite a fluorophore associated with the targeting agent;
receiving a signal indicating detection of a predetermined concentration of fluorescent emissions.

10. The method of claim 9, wherein:
the fluorescence detection instrument comprises eyewear; and
the receiving step further comprises the step of visualizing an image having information on the position and the relative intensity of the fluorescent emissions within the animal.

11. The method of claim 8, wherein:
the fluorescent targeting agent comprises a purified antibody that binds specifically to TAG-72.

12. The method of claim 10, wherein:
the fluorescent targeting agent comprises a humanized anti-TAG-72 antibody, HuCC49ΔC_{H}2.

13. A system for detecting a target tissue in an animal, comprising:
a molecular targeting agent operably linked to a fluorescent unit; and
a fluorescence detection instrument comprising:
a light source emitting an excitation radiation capable of exciting the fluorescent unit across an intervening biological tissue; and
a detection unit capable of detecting fluorescent emissions from the fluorescent unit across the intervening biological tissues.

14. The system of claim 13, wherein:
the molecular targeting agent comprises a purified antibody that binds specifically to TAG-72.

15. The system of claim 13, wherein:
the molecular targeting agent comprises a humanized anti-TAG-72 antibody, HuCC49ΔC_{H}2.

16. The system of claim 13, wherein:
the fluorescence detection instrument is hand-held.

17. The system of claim 13, wherein:
the fluorescence detection instrument comprises a wearable unit to excite, detect, and visually report the fluorescent deposition of the targeting agent to the user.

18. The system of claim 17, wherein:
the wearable unit comprises an eyewear article.

19. The system of claim 13, wherein:
the fluorescence detection instrument comprises a camera with fluorescence filter, a multi-wavelength excitation light source, a head mount display (HMD), and a computer.

20. The system of claim 13, wherein:
the fluorescent unit comprises a nanoparticle.

21. The system of claim 13, wherein:
the fluorescent unit comprises a microbubble or a nanobubble.
